(19) United States
(12) Patent Application Publication
(10) Pub. No.: US 2026/0114937 A1
(43) Pub. Date: Apr. 30, 2026

(54) CUP ALIGNMENT SYSTEMS AND METHODS

(71) Applicant: OrthAlign, Inc., Irvine, CA (US)

(72) Inventors: Rafael Sierra, Rochester, MN (US); Orian Bortz, Beverly Hills, CA (US); Steven DeVincentis, Laguna Hills, CA (US); James Gannoe, Laguna Niguel, CA (US)

(21) Appl. No.: 19/391,679

(22) Filed: Nov. 17, 2025

Related U.S. Application Data
(63) Continuation of application No. 17/833,714, filed on Jun. 6, 2022, now abandoned.
Application No. 17/833,714 is a continuation of application No. PCT/US2020/063785, filed on Dec. 8, 2020.
(60) Provisional application No. 62/945,591, filed on Dec. 9, 2019.

(51) Int. Cl. A61B 34/20; A61B 34/00; A61F 2/46

(52) U.S. Cl. CPC A61B 34/20; A61B 34/25; A61F 2/4609; A61B 2034/2048; A61F 2002/4633; A61F 2002/4668

(57) ABSTRACT

A system can include a module to measure mobility, such as pre-operative pelvic mobility, for surgical planning. The module can include one or more inertial sensors that can be positioned relative to the anatomy of a patient. Hip navigation systems can guide an acetabular cup to patient-specific target angles, based in part, on the pre-operative pelvic mobility of the patient.

### INCORPORATION BY REFERENCE TO ANY PRIORITY APPLICATIONS

This application is a continuation of U.S. patent application Ser. No. 17/833,714, filed Jun. 6, 2022, which is a continuation of International Application No. PCT/US2020/063785, filed Dec. 8, 2020, which claims the benefit under 35 U.S.C. § 119 (e) to U.S. Provisional Application No. 62/945,591, filed Dec. 9, 2019, which are hereby incorporated by reference in their entirety. Any and all applications for which a foreign or domestic priority claim is identified in the Application Data Sheet as filed with the present application is hereby incorporated by reference in its entirety under 37 CFR 1.57.

### BACKGROUND

### Field

This application is directed to the field of hip replacement, and particularly to surgical tools and methods for determining pelvic mobility pre-operatively. This application is also directed toward determining or recommending patient specific target angles for cup placement. This application is also directed to the use of collecting kinematic or positioning data with inertial sensors.

### Description of the Related Art

Hip replacement surgery is common and getting more common by the year. One persistent issue with hip replacement is poor placement of the cup component of the prosthetic hip joint. For example, the cup is optimally placed in a specific abduction and anteversion orientations. While an acceptable window of deviation from the optimal abduction and anteversion angles has been observed in clinical practice, for several reasons an unacceptably high percentage of patients have the cup of the artificial hip joint out of this window.

Unfortunately, misalignment can lead to dislocation of the hip as soon as within one year of the implantation procedure. This is particularly problematic because recovery from a hip procedure can take many months. Patients undergoing a revision so soon after the initial implantation will certainly be dissatisfied with their care, being subject to addition redundant surgery. Of course, all surgery carries some degree of risk. These poor outcomes are unsatisfactory for patients and are inefficient for the healthcare system as a whole.

### SUMMARY

There is a need for improved systems and methods for determining pelvic mobility and other patient specific kinematic data pre-operatively. There is also a need for improved systems and methods for improving alignment of hip components with a patient's anatomy during a hip replacement procedure by utilizing patient specific kinematic data. These improved systems and methods can involve use of pre-operative inertial sensors to determine pelvic mobility. The sensors can provide valuable information to a surgeon for use in selecting a patient-specific target. These improved systems and methods can involve improving patient specific target angles for cup placement. These improved systems and methods can involve inertial navigation of the cup inserter to the patient specific target angles during a hip replacement surgery. These improved systems and methods can involve adjustment of anteversion cup angles based on pre-operative kinematic information. These sensors can also provide valuable information on patient positioning during a surgical procedure.

In some embodiments, a system is provided. The system can include a module comprising one or more inertial sensors configured to be positioned relative to the anatomy of a patient. In some embodiments, the module is configured to measure mobility for surgical planning.

In some embodiments, the module comprises a biocompatible adhesive configured to adhere objects to the skin of the patient. In some embodiments, the module is embedded in a garment. In some embodiments, the one or more inertial sensors comprise an accelerometer. In some embodiments, the one or more inertial sensors comprise a gyroscope. In some embodiments, the module is configured to transmit data from the module to an external output device. In some embodiments, the external output device is a smartphone. In some embodiments, the system can include the smartphone. In some embodiments, the smartphone is configured to receive patient specific data obtained from the module. In some embodiments, the external output device is a surgical orientation device configured to be used during a surgical operation. In some embodiments, the system can include the surgical orientation device. In some embodiments, the surgical orientation device is configured to receive patient specific data obtained from the module.

In some embodiments, a system is provided. The system can include a surgical orientation device comprising an inertial sensor. In some embodiments, the surgical orientation device configured to facilitate navigation of an acetabular cup to the desired target angle. In some embodiments, the surgical orientation device detects orientation and rotation of the device relative to a reference frame. In some embodiments, the surgical orientation device is configured to receive one or more mobility measurements for surgical planning.

In some embodiments, the system can include an orientation sensing device. In some embodiments, the surgical orientation device comprises a transmitter for sending data or receiving data from one or more sensors of the orientation sensing device. In some embodiments, the surgical orientation device comprises a transmitter for sending data or receiving data from a module, wherein the module is configured to measure mobility. In some embodiments, the surgical orientation device comprises a transmitter for sending data or receiving data an external output device. In some embodiments, the external output device is a smartphone. In some embodiments, the smartphone is configured to receive patient specific data obtained from a module.

In some embodiments, a method of determining patient mobility is provided. The method can include positioning a module comprising one or more inertial sensors relative to the anatomy of a patient. The method can include measuring mobility with the module.

In some embodiments, the mobility is pelvic mobility. In some embodiments, positioning the module comprises positioning the module on the sacrum of the patient. In some embodiments, positioning the module comprises positioning the module on a femur of the patient. In some embodiments, positioning the module comprises positioning the module on the spine of the patient. In some embodiments, positioning the module comprises positioning the module on an ASIS point of the patient. In some embodiments, positioning the module comprises positioning the module on a bony prominence identified by palpation of the skin of the patient. In some embodiments, positioning the module comprises positioning the module on the skin of the patient over the underlying anatomy of the patient. In some embodiments, positioning the module comprises positioning the module on the pubis of the patient. In some embodiments, positioning the module comprises positioning the module on a palpable location on the body of the patient. In some embodiments, positioning the module comprises positioning the module on the lower back of the patient. In some embodiments, positioning the module comprises positioning the module on the upper back of the patient. In some embodiments, positioning the module comprises positioning the module on the neck of the patient. In some embodiments, positioning the module comprises positioning the module on the pelvis of the patient. In some embodiments, positioning the module comprises positioning the module on a tibia of the patient. In some embodiments, positioning the module comprises positioning the module on a vertebra of the patient. In some embodiments, positioning the module comprises positioning the module on the sternum of the patient. In some embodiments, positioning the module comprises positioning the module on a rib of the patient. In some embodiments, positioning the module comprises positioning the module on a cranial bone of the patient. In some embodiments, positioning the module comprises positioning the module on a facial bone of the patient. In some embodiments, positioning the module comprises positioning the module on a humerus of the patient. In some embodiments, positioning the module comprises positioning the module on a scapula of the patient. In some embodiments, positioning the module comprises positioning the module on a clavicle of the patient. In some embodiments, positioning the module comprises positioning the module on an ulna of the patient. In some embodiments, positioning the module comprises positioning the module on a radius of the patient. In some embodiments, positioning the module comprises positioning the module on a carpal bone of the patient. In some embodiments, positioning the module comprises positioning the module on a fibula of the patient. In some embodiments, positioning the module comprises positioning the module on a tarsus bone of the patient. In some embodiments, positioning the module comprises positioning the module on a metatarsal of the patient. In some embodiments, positioning the module comprises positioning the module on the patient for one or more hours. In some embodiments, positioning the module comprises positioning the module on the patient for one or more days. In some embodiments, positioning the module comprises positioning the module on the patient for one or more weeks. In some embodiments, the one or more inertial sensors comprise an accelerometer. In some embodiments, the one or more inertial sensors comprise a gyroscope. In some embodiments, the method can include transmitting data from the module to an external output device. In some embodiments, the external output device is a smartphone.

In some embodiments, a method of positioning a medical prosthesis is provided. The method can include reviewing pre-operative pelvic mobility measurements, wherein the measurements were collected with a module comprising one or more inertial sensors. The method can include determining patient specific target angles in view of the pre-operative pelvic mobility. The method can include aligning a cup to the patient specific target angles. The method can include impacting the cup.

In some embodiments, the one or more inertial sensors comprise an accelerometer. In some embodiments, the one or more inertial sensors comprise a gyroscope. In some embodiments, the method can include transmitting data from the module to an external output device. In some embodiments, the external output device is a smartphone. In some embodiments, the method can include determining a change in leg length before and after cup placement. In some embodiments, the method can include determining a change in leg length between the two legs. In some embodiments, the method can include determining a change in joint offset before and after cup placement. In some embodiments, the method can include projecting the pattern light onto the leg of the patient before impacting the cup and recording the incidence of light. In some embodiments, the method can include projecting the pattern light onto the leg of the patient after impacting the cup and repositioning the leg to align the recording of the incidence of light with the pattern of light. In some embodiments, the method can include recording a point before and after impacting the cup. In some embodiments, the method can include establishing a vertical plane. In some embodiments, the method can include establishing a horizontal plane, wherein the vertical plane and the horizontal plane define a reference frame. In some embodiments, the patient specific target angles are measured relative to the vertical plane. In some embodiments, the method can include establishing a reference plane. In some embodiments, the method can include establishing a reference plane including positioning an indicator to contact a first point, recording the position and/or orientation of the indicator when the indicator is contacting the first point, positioning the indicator to contact a second point, and recording the position and/or orientation of the indicator when the indicator is contacting the second point. In some embodiments, establishing a reference plane further comprises positioning the indicator to contact a third point and recording the position and/or orientation of the indicator when the indicator is contacting the third point. In some embodiments, the first point, the second point, and the third point define an anterior pelvic plane. In some embodiments, establishing a reference plane further comprises positioning the indicator horizontally. In some embodiments, the first point and the second point define a line intersecting contralateral ASIS.

In some embodiments, a method is provided. The method can include positioning a module comprising one or more inertial sensors relative to the anatomy of a patient. The method can include measuring patient specific pelvic mobility with a module comprising one or more inertial sensors. In some embodiments, the patient specific pelvic mobility is reviewed for determining at least a portion of the surgical plan.

In some embodiments, the patient specific pelvic mobility is reviewed for determining target angles. In some embodiments, the patient specific pelvic mobility is reviewed for determining the type of implant. In some embodiments, the patient specific pelvic mobility is reviewed for determining the type of instrumentation. In some embodiments, the patient specific pelvic mobility is reviewed for determining leg length. In some embodiments, the patient specific pelvic mobility is reviewed for determining joint offset. In some embodiments, the one or more inertial sensors comprise an accelerometer. In some embodiments, the one or more inertial sensors comprise a gyroscope. In some embodiments, the method can include transmitting data from the module to an external output device. In some embodiments, the external output device is a smartphone.

In some embodiments, a hip joint navigation system is provided. The hip joint navigation system can include a first inertial navigation device comprising one or more inertial sensors. The hip joint navigation system can include an indicator. The hip joint navigation system can include a second inertial navigation device comprising one or more inertial sensors. In some embodiments, the first inertial navigation device, the second inertial navigation device, or the first inertial navigation device and the second inertial navigation device are configured to align a cup to the patient specific target angles, wherein the patient specific target angle are determined based on pre-operative pelvic mobility.

In some embodiments, the system can include a module comprising one or more inertial sensors, wherein the module is configured to measure the pre-operative pelvic mobility. In some embodiments, the first inertial navigation device comprises a transmitter for sending data or receiving data from the module. In some embodiments, the second inertial navigation is configured to couple to an impactor. In some embodiments, the hip joint navigation system can include a universal impactor adaptor comprising a coupler, wherein the second inertial navigation is configured to couple to an impactor with the adaptor. In some embodiments, the hip joint navigation system can include an optical component.

In some embodiments, an orthopedic surgery guidance system is provided. The orthopedic surgery guidance system can include a module configured to generate an output indicating at least a portion of a surgical plan based on pre-operatively measured joint mobility. The orthopedic surgery guidance system can include a user interface configured to display information pertaining to the output during surgery.

In some embodiments, the system can include a module comprising one or more inertial sensors, wherein the module is configured to measure the pre-operative joint mobility. In some embodiments, the modules comprises a transmitter for sending data or receiving data therebetween. In some embodiments, the display indicates a type of implant to be used the orthopedic surgery. In some embodiments, the display indicates a use of a dual mobility hip implant. In some embodiments, the display indicates one or more target angles toward which a surgical instrument is to be aligned and/or one or more target positions toward which the surgical instrument is to be advanced. In some embodiments, the display indicates proximity of a surgical instrument to the one or more target angles and/or the one or more target positions. In some embodiments, the orthopedic surgery guidance system can include a reamer, the system comprising a module configured to provide an output indicative of position and/or orientation of the reamer relative to the one or more target angles and/or the one or more target positions and the user interface is configured to display the output indicative of position and/or orientation. In some embodiments, the orthopedic surgery guidance system can include an impactor, the system comprising a module configured to provide an output indicative of position and/or orientation of the impactor relative to the one or more target angles and/or the one or more target positions and the user interface is configured to display the output indicative of position and/or orientation. In some embodiments, the orthopedic surgery guidance system can include obtaining a reference frame. In some embodiments, the orthopedic surgery guidance system can include obtaining a reference frame by using an indicator to establish position and/or orientation of a point. In some embodiments, the orthopedic surgery guidance system can include obtaining a reference frame by using an indicator to establish orientation of an axis. In some embodiments, the orthopedic surgery guidance system can include obtaining a reference frame by using an indicator to establish position and/or orientation of an axis of gravity. In some embodiments, the orthopedic surgery guidance system can include obtaining a reference frame by using an indicator to establish position and/or orientation of a plane. In some embodiments, the orthopedic surgery guidance system can include obtaining a reference frame by using an indicator to establish position and/or orientation of a plane that approximates an anatomical plane of the patient. In some embodiments, the orthopedic surgery guidance system can include obtaining a reference frame by using an indicator to establish position and/or orientation of a plane that approximates the plane of an image of the patient.

In some embodiments, a method is provided. The method can include positioning a module comprising one or more inertial sensors relative to the anatomy of a patient. The method can include measuring patient specific pelvic mobility with a module comprising one or more inertial sensors. In some embodiments, the patient specific pelvic mobility is collected post-operatively.

In some embodiments, the method can include positioning a module comprising one or more inertial sensors relative to the anatomy of a patient pre-operatively; and measuring patient specific pelvic mobility with a module comprising one or more inertial sensors preoperatively. In some embodiments, the method can include comparing the pre-operative and post-operative measurements. In some embodiments, the pre-operative patient specific pelvic mobility is reviewed for determining at least a portion of the surgical plan. In some embodiments, the post-operative patient specific pelvic mobility is reviewed for determining at least a portion of the physical therapy plan.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments.

FIG. 1 illustrates a perspective view of a module in the hand of a patient.

FIG. 2 illustrates a view of components of a pre-operative planning system.

FIGS. 3A-3C illustrate perspective views of the module of FIG. 1 coupled to a patient.

FIG. 4 illustrates view of one or more modules of FIG. 1 in communication with an external output device.

FIGS. 5A-5B illustrate pre-operative methods.

FIG. 6 illustrates an embodiment of a workflow.

FIG. 7 illustrates a perspective view of a hip navigation system coupled to a pelvis to a patient.

FIG. 8 illustrates the patient positioning for the posterior hip approach.

FIG. 9 illustrates the projection of light from the optical component.

FIG. 10 illustrates a method of table registration.

FIG. 11 illustrates an embodiment of an impactor including the orientation sensing device of FIG. 7.

FIG. 12 illustrates an example of an x-ray that can be taken pre-operatively.

FIG. 13 illustrates the patient positioning for the anterior hip approach.

FIG. 14 illustrates a method to identify the Anterior Pelvic Plane.

FIG. 15 illustrates a method to identify an Adjusted Plane.

FIG. 16 illustrates side views of the pelvis, the Anterior Pelvic Plane, and the Adjusted Plane.

### DETAILED DESCRIPTION

A variety of systems and methods are discussed below that can be used to improve outcomes for patients by increasing the likelihood of proper placement of a medical prosthesis such as an acetabular cup. In some embodiments, pre-operative planning systems and methods are utilized to measure characteristics of the patient's anatomy. In some embodiments, intraoperative navigation systems and methods are utilized to guide a medical prosthesis to a desired angular placement relative to a reference plane.

### A. Pre-Operative Planning Systems for Anterior and Poster Approach

### 1. Pre-Operative Planning System

A variety of modules, systems, and methods for total hip arthroplasty (THA) are discussed below that can be used to improve outcomes for patients by increasing the likelihood of proper placement of a medical prosthesis such as an acetabular cup. These systems and methods can be focused on understanding the patient's anatomy, such as pelvic mobility pre-operatively and patient positioning during a patient procedure. These systems and methods can add a functionality of measuring, quantifying, and/or tracking characteristics of pelvic mobility or patient positioning. These sensor measurements inform the selection of a patient specific target abduction and anteversion angles for cup placement. These sensor measurements can enable a surgeon to determine the patient position during a surgical procedure. These sensor measurements can have a variety of uses to improve patient outcomes, as described herein.

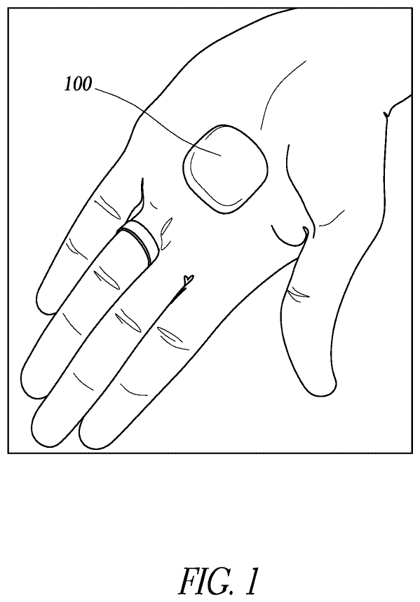

FIG. 1 illustrates a module 100 adapted to determine one or more characteristics of a patient. The module 100 can determine kinematic information related to the patient. In some embodiments, the module 100 is configured to measure pelvic mobility. In some embodiments, the module 100 can be used to measure mobility of another joint. The module 100 can be used to measure mobility between the pelvis and the spine. The module 100 can be used to measure mobility between the pelvis and at least one femur. In some embodiments, the module 100 is configured to measure pelvic mobility or some values commensurate with pelvic mobility. While characterizing pelvic mobility is described herein, the modules 100 can be used to measure any characteristic of movement or positioning of the patient. The modules can be utilized with any portion of the anatomy including the spine, pelvis, femur, tibia, vertebrae, sternum, ribs, cranial bones, facial bones, humerus, scapula, clavicle, ulna, radius, carpal bones, fibula, tarsus bones, metatarsals, and/or any other bone or region of the human body.

The module 100 can determine the position or orientation of a portion of the patient's anatomy relative to another portion of the patient's anatomy. The module 100 can determine the position or orientation of a portion of the patient's anatomy relative to a coordinate system. In some embodiments, the module 100 is configured to determine the position or orientation of a portion of the patient's anatomy relative a gravitational axis. In some embodiments, the module 100 is configured for dynamic measurements. In some embodiments, the module 100 is configured for static measurements.

The module 100 can be used pre-operatively to determine a characteristic of the patient. In some embodiments, the pre-operative information can be utilized by the surgeon during the surgical procedure. In some embodiments, the pre-operative information can be utilized by the surgeon to determine target cup angles. In some embodiments, the pre-operative information can be utilized by the surgeon to modify the surgical plan based on the patient specific data. In some embodiments, the pre-operative information can be utilized by the surgeon to adjust the cup angles based on the patient specific data. The module 100 can be used pre-operatively to provide data for a later surgical procedure. In some embodiments, the module 100 can inform a surgeon about a target. In some embodiments, the module 100 can inform a surgeon to modify a target angle determined from static images such as x-rays. In some embodiments, the module 100 can recommend an adjustment to the target angles. The surgeon can determine the patient specific target angles. In some embodiments, the pre-operative information can be utilized in other ways, as described herein.

The patient specific target angles can be an input in to a system that allows navigation to the patient specific angles. In some methods, the module 100 can be used intra-operatively. The module 100 can determine patient positioning. The module 100 can provide additional data related to a reference frame. The module 100 can facilitate alignment of the anatomy of the patient. In some embodiments, the module 100 can be utilized in other ways, as described herein.

The module 100 can be used post-operatively to determine a characteristic of the patient. In some embodiments, the post-operative information can be utilized by the surgeon or physician to determine the outcome of the surgical procedure. In some embodiments, the post-operative information can be utilized by the surgeon or physician to modify the behavior of the patient. In some embodiments, the post-operative information can be utilized by the surgeon or physician to modify the post-operative plan for the patient. In some embodiments, the post-operative information can be utilized by the surgeon or physician to modify post-operative physical therapy. In some embodiments, the post-operative information can be utilized in other ways, as described herein.

In some embodiments, the module 100 can provide information enabling the surgeon to categorize the patient. In some embodiments, the module 100 can provide information enabling the surgeon to modify population target angles to patient specific target angles. In some embodiments, the patient specific information from the module 100 can inform the surgeon to alter the anterversion angle for cup placement. In some embodiments, the patient specific information from the module 100 can inform the surgeon to alter the abduction angle for cup placement. In some embodiments, the patient specific information from the module 100 can inform the surgeon to modify the target angles for cup placement. In some embodiments, the module 100 is configured to facilitate the determination of patient specific target angles for cup placement.

In some embodiments, the patient specific information from the module 100 can guide the surgeon in prosthesis selection. The patient specific information from the module 100 can inform the surgeon to select a prosthesis from two or more prostheses. In some embodiments, the patient specific information from the module 100 can inform the surgeon to select a dual mobility implant.

In some embodiments, the module 100 can transmit data to be displayed. The module 100 can be configured to show values of pelvic angles. The module 100 can be configured to show a recommendation for patient specific target angles. The module 100 can be configured to show kinematic data. The module 100 can be configured to show positioning data. The module 100 can be configured to determine a recommendation. The module 100 can be configured to process data into a form readable by the surgeon. The module 100 can be configured to process data into a bulleted list. The module 100 can be configured to process data into a graph. The module 100 can be configured to process data into a verbal recommendation.

The module 100 can comprise a compact, generally hand-held and/or portable device. The module 100, as described herein, can be used alone or in conjunction with other devices, components, and/or systems. In some embodiments, the module 100 can be used in conjunction with a surgical procedure. The module 100 can be used pre-operatively, post-operatively, and/or during the surgical procedure. In uses of the module 100 involving a surgical procedure, the module 100 can be used in combination with a posterior approach for total hip arthroplasty. In uses of the module 100 involving a surgical procedure, the module 100 can be used in combination with an anterior approach for total hip arthroplasty.

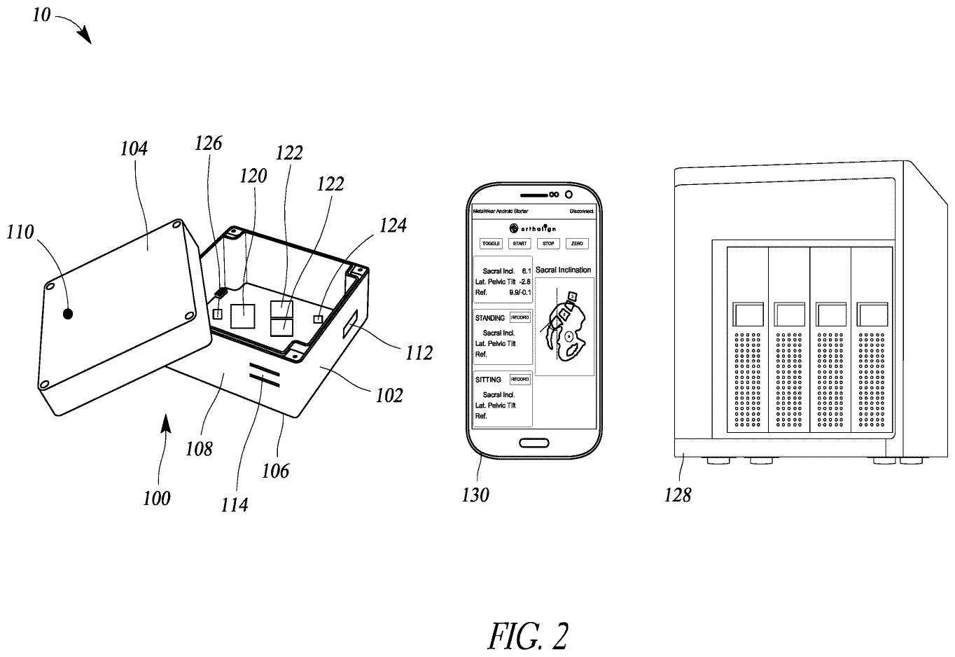

The module 100 can be used in combination with other components, thereby forming a system 10. In some methods, the system 10 is utilized as a pre-operative planning system. In some methods, the system 10 is utilized as a post-operative planning system. In some methods, the system 10 is utilized during a surgical procedure. FIG. 2 illustrates an embodiment of the system 10. The system 10 can include any additional component. The system 10 can omit any component described herein. The system 10 can include one or more separate components. In some embodiments, two or more components of the system 10 can be integrated into a single component.

The module 100 can take on any shape or form. In some embodiments, the module 100 can comprise a three-dimension shape such as a sphere, cone, cylinder, cube, cuboid, triangular prism, pyramid, torus, helix, or any other three-dimensional shape. In some embodiments, the module 100 can comprise a two-dimensional shape such as circle, triangle, square, rectangle, semi-circle, oval, or any other two-dimensional shape. In some embodiments, the module 100 can comprise an amorphous shape. In the illustrated embodiment, the module 100 can comprise a generally rectangular-shaped structure, but other configurations are contemplated. In some embodiments, the module 100 can comprise a rigid structure. In some embodiments, the module 100 can comprise a flexible substrate. In some embodiments, the module 100 can comprise a shape or form that conforms to the skin of the patient. In some embodiments, the module 100 can have an outer housing 102. The outer housing 102 can be portable. The outer housing 102 can be comprised, at least in part, of plastic including but not limited to ABS, polycarbonate, or other suitable material. The module 100 can be configured for hand-held use. The outer housing 102 can include a front surface 104 that faces generally away from the body of the user. The outer housing 102 can include a back surface 106 that faces toward the body of the user. The outer housing 102 can include one or more lateral surfaces 108.

In some embodiments, the module 100 is reusable. The module 100 can be configured for multiple uses by the same patient. The module 100 can be configured for multiple uses by different patients. In some embodiments, the module 100 can be cleaned or sterilized between uses. The module 100 can include an internal source of power such as a battery. The module 100 can be reusable as long as the power source is functional. The module 100 can include a replaceable battery or power source. The module 100 can be configured to be opened by the patient or physician to replace the battery or power source. The module 100 can include a rechargeable battery or power source. The module 100 can be configured to be coupled to a charger to resupply power to the battery or power source.

In some embodiments, the module 100 is disposable. The module 100 can be single use. In some embodiments, the module 100 can be reused by placing the module 100 within a disposable housing. This arrangement can maximize reuse of internal components while maintaining the cleanliness of the module 100. The disposable outer housing can include, or be releasably attached to, the module 100. The disposable outer housing can be manufactured and packaged in a sterile state and can provide a sterile barrier between the reusable components inside the module 100 and the outside environment. Thus, once the module 100 has been used, the disposable outer housing can be discarded or destroyed, and the interior, reusable components can be used again.

The module 100 can comprise an indicator 110. The indicator 110 can be located on the front surface 104. The indicator 110 can be located on the lateral surfaces 108. The indicator 110 can be a light or LED indicating that the module 100 is turned on. The indicator 110 can be a light or LED indicating that the module 100 is sensing movement. The indicator 110 can be a separate component from the outer housing 102 or can be integrated on or within the outer housing 102. In some embodiments, the indicator 110 is a display. The display can be sized such that a user can readily read numbers, lettering, and/or symbols on the display while performing a medical procedure. The indicator 110 can provide a warning to the user when a particular condition occurs. The indicator 110 can comprise a visible output, such as one or more LED status or notification lights, for example, to indicate low battery level, an error condition, etc. The indicator 110 can comprise different patterns, tones, cadences, durations, and/or frequencies to signify different conditions or events.

The module 100 can comprise a user input device 112. The user input device 112 can be located on the front surface 104. The user input device 112 can be located on the lateral surfaces 108. The user input device 112 can comprise one or more buttons. The user input device 112 can comprise one or more switches. The user input device 112 can comprise one or more scroll wheels. The user input device 112 can be activated, for example, by a finger, hand, and/or instrument to select a mode or modes of operation of the module 100. The user input device 112 can be a separate component from the outer housing 102 or can be integrated on or within the outer housing 102. In some embodiments, the user input device 112 is a separate component from the housing 102. For example, the user input device 112 can comprise a remote input device coupled to the module 100 via a wired or wireless connection. The user input device 112 can receive a signal, such as a signal to turn on or record measurements. The user input device 112 can receive a signal to turn off. The user input device 112 can receive a signal indicating the duration or time period that the module 100 records measurements. The module 100 can include a means to receive instructions.

The module 100 can include a means to couple to the patient. The module 100 can be removably coupled to the patient. In some embodiments, the back surface 106 of the module 100 can include a means to couple to the patient. In some embodiments, the back surface 106 comprises an adhesive. The adhesive can be any biocompatible adhesive configured to adhere objects to the skin of the patient. In some embodiments, an adhesive can be coupled or adhered to the back surface 106. The back surface 106 can include an attachment structure or structures. The attachment structures can facilitate attachment of the module 100 to another device or the patient. In some methods, the module 100 can be positioned on a portion of the patient's body with little soft tissue. In some methods, the module 100 can be positioned on a portion of the patient's body close to the underlying anatomy. In some methods, the module 100 can be positioned on a portion of the patient's body at one or more palpable locations.

In some embodiments, the module 100 is coupled to a wrap or band. In some embodiments, the module 100 can be embedded in a fabric. In some embodiments, the module 100 can be embedded in a garment. The module 100 can be monolithically formed with a fabric or garment. The module 100 can be integrated with a fabric or garment. The garment can be tight-fitting. The garment can position the module 100 relative to the patient's anatomy. The garment can be configured to position one or more modules 100 relative to the pelvis. In some embodiments, the garment is a pair of shorts or pants. In some embodiments, the garment is a t-shirt. The garment can be made of any tight-fitting material such as spandex, polyester, polypropylene, nylon, cotton, bamboo, neoprene, or any other material. In some embodiments, the module 100 is coupled to a garment configured to be worn by the patient. In some embodiments, the module 100 is wearable. In some embodiments, the module 100 can wrap around a portion of the body of the patient. In some embodiments, the module 100 is a sleeve or strap.

In some embodiments, the module 100 is configured to be external to the body of the patient. The module 100 can couple to the skin of the patient. The module 100 is configured to be easily coupled to the body of the patient. The module 100 is configured easily removed from the body of the patient.

The module 100 can be coupled to the patient for a period of time. In some methods, the module 100 is coupled to the patient for one or more minutes, one or more hours, one or more days, one or more weeks, or any range of the foregoing values. In some methods, the module 100 is coupled to the patient for 5 minutes, 10 minutes, 15 minutes, 20 minutes, 25 minutes, 30 minutes, 35 minutes, 40 minutes, 45 minutes, 50 minutes, 55 minutes, 60 minutes, or any range of two of the foregoing values. In some methods, the module 100 is coupled to the patient for a portion of a clinical appointment. In some methods, the module 100 is coupled to the patient for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 21 hours, 22 hours, 23 hours, 24 hours, 36 hours, 48 hours, 60 hours, 72 hours, or any range of two of the foregoing values. In some methods, the module 100 is coupled to the patient for a portion of a hospital or in-patient stay. In some methods, the module 100 is coupled to the patient for 1 day, 2 days, 3 days, 4 days, 5 days, 6 days, 7 days, 8 days, 9 days, 10 days, 14 days, 21 days, 28 days, or any range of two of the foregoing values. In some methods, the module 100 is coupled to the patient for a portion of pre-operative monitoring. In some methods, the module 100 is coupled to the patient for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or any range of two of the foregoing values. In some methods, the module 100 is coupled to the patient for diagnostic purposes. In some methods, the module 100 is coupled to the patient for 1 month, 2 months, 3 months, 4 months, 5 months, 6 months, 7 months, 8 months, 9 months, 10 months, 11 months, 12 months, or any range of two of the foregoing values. In some methods, the module 100 is coupled to the patient to monitor a condition. In some methods, the module 100 is coupled to the patient to monitor a change or deterioration in condition. In some methods, the module 100 is coupled to the patient continuously during the period of time. In some methods, the module 100 is coupled to the patient sporadically during the period of time. In some methods, the module 100 is coupled to the patient during certain activities during the period of time. In some methods, the module 100 is coupled to the patient during waking hours during the period of time. In some methods, the module 100 is coupled to the patient during movements during the period of time. In some methods, the module 100 is coupled to the patient on a rotational basis, e.g., such as 1 hour every Monday or 1 day every week. In some methods, the module 100 is coupled to the patient according to a schedule. In some methods, the module 100 is coupled to the patient to record the desired amount of data over the desired period of time. In some methods, the module 100 is coupled to the patient for the duration of one or more movements. In some methods, the module 100 is coupled to the patient for the duration of one or more positions. In some methods, the module 100 is coupled to the patient for the duration of the surgical procedure.

The module 100 can include gripping feature or features 114 for facilitating handling of the module 100. In some embodiments, the gripping feature or features 114 comprise grooves, or channels, along a portion of the module 100. The gripping feature or features 114 can be located on the front surface 104. The gripping feature or features 114 can be located on the back surface 106. The gripping feature or features 114 can be formed, for example, from protruding portions from the lateral surfaces 108. The gripping feature or features 114 can extend partially, or entirely, along the lateral surfaces 108 of the module 100.

In some embodiments, the module 100 can include one or more functional components configured to sense position, orientation, or movement. The module 100 can comprise an electronic control unit 120 that communicates with one or more sensors 122. In some embodiments, the one or more sensors 122 comprise one or more inertial sensors. The module 100 can comprise a power supply 124. The module 100 can comprise internal memory 126. FIG. 2 illustrates the electronic control unit 120, the one or more sensors 122, and the power supply 124. The electronic control unit 120 can be a separate component from the outer housing 102 or can be integrated on or within the outer housing 102. The one or more sensors 122 can be separate components from the outer housing 102 or can be integrated on or within the outer housing 102. The power supply 124 can be a separate component from the outer housing 102 or can be integrated on or within the outer housing 102.

The module 100 can be used in combination with other components, thereby forming the system 10 as shown in FIG. 2. The system 10 can include external memory 128. The module 100 can communicate with the external memory 128. The system 10 can include an external output device 130. The module 100 can communicate with the external output device 130. In some embodiments, the external output device 130 can include the external memory 128. The external memory 128 can be a separate component from the external output device 130 or can be integrated on or within the external output device 130. The system 10 can include includes the external output device 130 with which a user can interact. The external output device 130 can allow a surgeon, medical personnel, and/or other user to operate the module 100 with ease, efficiency, and accuracy.

In some embodiments, the electronic control unit 120 receives input from the one or more sensors 122. The electronic control unit 120 can control and/or transmit output to the external memory 128. The electronic control unit 120 can control and/or transmit output to the external output device 130. The electronic control unit 120 can be configured to receive and send electronic data. The electronic control unit 120 can be configured to perform calculations based on received electronic data. The electronic control unit 120 can include a transmitter. The electronic control unit 120 can transfer data wirelessly, using Bluetooth™, Bluetooth Low Energy™, Wifi® or other standard wireless telemetry protocol. The electronic control unit 120 can include a BlueTooth™ radio. The electronic control unit 120 can include Bluetooth Low Energy™ radio.

In certain embodiments, the electronic control unit 120 can be configured to convert the electronic data from a machine-readable format to a human readable format for presentation on the external output device 130. The electronic control unit 120 can comprise one or more processors, program logic, or other substrate configurations representing data and instructions. The electronic control unit 120 can comprise controller circuitry, processor circuitry, processors, general purpose single-chip or multi-chip microprocessors, digital signal processors, embedded microprocessors, microcontrollers and/or the like. The electronic control unit 120 can comprise conventional address lines, conventional data lines, and one or more conventional control lines. The electronic control unit 120 can comprise an application-specific integrated circuit (ASIC) or one or more modules configured to execute on one or more processors. The electronic control unit 120 can comprise comprises a microcontroller.

The electronic control unit 120 can communicate with internal memory 126 to retrieve and/or store data. The electronic control unit 120 can communicate with external memory 128 to retrieve and/or store data. The electronic control unit 120 can communicate with internal memory 126 and/or external memory 128 to retrieve program instructions for software and/or hardware. The internal memory 126 and the external memory 128 can include random access memory (“RAM”), such as static RAM, for temporary storage of information and/or read only memory (“ROM”), such as flash memory, for more permanent storage of information. The external memory 128 can be integrated into a cloud database. The external output device 130 can retrieve data from a cloud database. The one or more modules 100 can interact with the external memory 128 via cloud integration. The external memory 128 can be a server.

In some embodiments, the electronic control unit 120 can be configured to provide continuous real-time data to the external output device 130. In some embodiments, the electronic control unit 120 can be configured to provide continuous data collection over a range of time. In some embodiments, the electronic control unit 120 can be configured to generate a plurality of data points during patient movement (e.g., 10 data points, 100 data points, 500 data points, 1000 data points, or any range of the foregoing values). In some embodiments, the electronic control unit 120 can be configured to generate a plurality of data points over a period of time (e.g., 10 data points per minute, 100 data points per minute, 500 data points per minute, 1000 data points per minute, or any range of the foregoing values).

The electronic control unit 120 can be configured to receive the real-time data from the one or more sensors 122. The electronic control unit 120 can be configured to use the sensor data to determine, estimate, and/or calculate a pelvic mobility or measure values proportional to pelvic mobility of the patient. The electronic control unit 120 can be configured to use the sensor data to determine patient positioning. The mobility information can be used to determine patient specific target angles for a hip replacement surgery. The mobility information can be used to classify patients to modify population average target angles. In some embodiments, the one or more sensors 122 facilitate determination of pelvic mobility and/or patient positioning during a preparation procedure performed before the orthopedic surgery.

In some embodiments, the one or more sensors 122 can comprise at least one orientation sensor configured to provide real-time data to the electronic control unit 120 related to the motion, orientation, and/or position of the corresponding anatomy of the patient. In some embodiments, the one or more sensors 122 can comprise at least one gyroscopic sensor, accelerometer sensor, tilt sensor, magnetometer and/or other similar device or devices. In some embodiments, the one or more sensors 122 can comprise optical navigation sensors. In some embodiments, the one or more sensors 122 can comprise non-inertial sensors. In some embodiments, the one or more sensors 122 can comprise any sensor that can determine position. In some embodiments, the one or more sensors 122 can comprise any sensor that can determine orientation. In some embodiments, the one or more sensors 122 can comprise any sensor that can track motion. The one or more sensors 122 can be configured to measure, and/or facilitate determination of, the mobility of the anatomy of the patient underlying the one or more sensors 122. In some embodiments, the one or more sensors 122 can be configured to provide measurements relative to a reference point(s), line(s), plane(s), and/or gravitational zero. Gravitational zero, as referred to herein, refers generally to an orientation in which an axis of the sensor is perpendicular to the force of gravity, and thereby experiences no angular offset, for example tilt, pitch, roll, or yaw, relative to a gravitational force vector. In some embodiments, the one or more sensors 122 can be configured to provide measurements for use in dead reckoning or inertial navigation systems.

In some embodiments, the one or more sensors 122 can comprise one or more accelerometers that measure the static acceleration of the patient's anatomy due to gravity. For example, the accelerometers can be used as tilt sensors to detect rotation of the patient's anatomy about one or more of its axes. The one or more accelerometers can comprise a dual axis accelerometer, which can measure rotation about two axes of rotation. The one or more accelerometers can comprise a three-axis accelerometer, which can measure rotation about three axes of rotation. The changes in orientation about the axes of the accelerometers can be determined relative to gravitational zero and/or to any coordinate system described herein.

The output signals of one or more accelerometers can comprise digital signals. The one or more accelerometers can output digital signals over a standard digital interface. The one or more accelerometers can output digital signals over any protocol including Inter-integrated Circuit (I2C). The one or more accelerometers can output digital signals over any commination interface including Serial Peripheral Interface (SPI). The one or more accelerometers can output digital signals over any protocol such as Universal Asynchronous Receiver/Transmitter (UART) or CAN protocol. The output signals of one or more accelerometers can comprise analog voltage signals. The output voltage signals for each axis can fluctuate based on the fluctuation in static acceleration as the accelerometer changes its orientation with respect to the gravitational force vector. In certain embodiments, an accelerometer experiences static acceleration in the range from −1 g to +1 g through 180 degrees of tilt, with −1 g corresponding to a −90 degree tilt, Og corresponding to a zero degree tilt, and +1 g corresponding to a +90 degree tilt. The acceleration along each axis can be independent of the acceleration along the other axis or axes.

Multi-axis accelerometers can be conceptualized as having a separate accelerometer sensor for each of its axes of measurement, with each sensor responding to changes in static acceleration in one plane. In certain embodiments, each accelerometer sensor is most responsive to changes in tilt (i.e., operates with maximum or optimum accuracy and/or resolution) when its sensitive axis is substantially perpendicular to the force of gravity (i.e., when the longitudinal plane of the accelerometer sensor is parallel to the force of gravity) and least responsive when the sensitive axis is parallel to the force of gravity (i.e., when the longitudinal plane of the accelerometer sensor is perpendicular to the force of gravity). In some embodiments, the one or more sensor 122 can be mounted to be offset at an angle to improve accuracy of the one or more sensor 122.

In some embodiments, the one or more sensors 122 comprise at least one single- or multi-axis gyroscope sensor and at least one single- or multi-axis accelerometer sensor. The one or more sensors 122 can comprise a three-axis gyroscope sensor, or three gyroscope sensors. The one or more sensors 122 can comprise a three-axis accelerometer, or three accelerometer sensors. The one or more sensors 122 can provide position and orientation measurements for all six degrees of freedom of the module 100. In some embodiments, the one or more sensors 122 provide an inertial navigation or dead reckoning system to continuously calculate the position, orientation, and velocity of the module 100 without the need for external references.

In some embodiments, the one or more sensors 122 comprise one or more accelerometers and at least one magnetometer. The magnetometer can be configured to measure a strength and/or direction of one or more magnetic fields in the vicinity of the module 100. The magnetometer can advantageously be configured to detect changes in angular position about a horizontal plane. In some embodiments, the one or more sensors 122 comprise one or more sensors capable of determining distance measurements. The one or more sensors 122 can be in electrical communication, wired or wireless, with an emitter element mounted at the end of a measurement indicator. In certain embodiments, the electrical control unit 120 can be configured to determine the distance between the sensor and emitter, for example, an axial length of a measurement indicator corresponding to a distance to an anatomical landmark. In some embodiments, the indicator can be a probe.

In some embodiments, the module 100 can collect data relative to a frame of reference. In some embodiments, the module 100 can collect data relative to a coordinate system. The coordinate system can include two perpendicular planes. The coordinate system can include three perpendicular planes. The coordinate system can include two perpendicular axes. The coordinate system can include three perpendicular axes. The one or more modules 100 can register to a global coordinate system. The global coordinate system can be utilized in one or more method steps. The global coordinate system can be utilized with cup navigation. The global coordinate system can be anatomically based. The global coordinate system can be determined pre-operatively. The global coordinate system can be determined intra-operatively. The global coordinate system can be determined from imaging techniques such as x-rays. The global coordinate system include one or more anatomical axes. The global coordinate system can include one or more anatomical planes. The global coordinate system can include one or more planes determined from gravity.

The module 100 can synchronize with the global coordinate system. The module 100 can determine orientation of the module 100 within the global coordinate system. The module 100 can utilize the global coordinate system to preserve accuracy. The one or more modules 100 can determine orientation relative to a coordinate system including the Anterior Pelvic Plane. The one or more modules 100 can determine orientation relative to a coordinate system including the Adjusted Plane. The one or more modules 100 can determine orientation relative to a coordinate system that includes a plane that approximates the Anterior Pelvic Plane. The one or more modules 100 can determine orientation relative to a coordinate system that includes a vertical plane. The one or more modules 100 can determine orientation relative to a coordinate system that includes a horizontal plane. The one or more modules 100 can determine orientation relative to a coordinate system including the axis of gravity. The one or more modules 100 can determine orientation relative to a coordinate system including an origin point. The one or more modules 100 can determine orientation relative to a coordinate system related to a standing lateral film or x-ray. The one or more modules 100 can determine orientation relative to a pre-determined reference frame.

The output of the module 100 can be correlated to a reference frame used for navigation. The output of the module 100 can be correlated to a reference frame used for cup placement. The measurements of the module 100 can be displayed. The measurements of the module 100 can be displayed in the same coordinate system that angles for anteversion are displayed. The measurements of the module 100 can be displayed in the same coordinate system that angles of pelvic tilt are displayed. The measurements of the module 100 can be correlated with the coordinate system by an algorithm. The measurements of the module 100 can be correlated with the coordinate system that was determined by registration of points. The measurements of the module 100 can be correlated with the coordinate system that was determined by registration of the table.

The power supply 124 can comprise one or more power sources configured to supply power to the electronic control unit 120. In some embodiments, the power supply 124 comprises one or more rechargeable or replaceable batteries. In some embodiments, the power supply 124 comprises one or more capacitive storage devices such as one or more capacitors or ultracapacitors. In some embodiments, power can be supplied by other wired and/or wireless power sources. The electronic control unit 120 can be configured to monitor the battery level if a battery is used for the power supply 124. Monitoring the battery level can advantageously provide advance notice of power loss. In certain embodiments, the module 100 can comprise a timer configured to cause the module 100 to temporarily power off after a predetermined period of inactivity and/or to permanently power off after a predetermined time-out period.

The module 100 can comprise one or more circuit boards and/or other circuitry capable of installation within the module 100. The mounting of the one or more sensors 122 can enable the one or more sensors 122 to operate in the regions of maximum or optimum sensitivity, accuracy, and/or resolution. The particular mounting offset angle can be selected based on a range of motion of the module 100 during a particular expected movement of the patient.

In some embodiments, the electronic control unit 120 can convert an analog voltage output signal of one or more sensors 122 into an angle degree measurement for presentation on the external output device 130. Each of the steps can be implemented using hardware and/or software. For each axis of rotation measured (e.g., pitch and roll), the one or more sensors 122 can continuously output an analog voltage signals. The signal conditioning circuitry of the module 100 can filter the analog output voltage signal (e.g., with a low pass filter) to remove noise from the signal. The signal conditioning circuitry amplifies, or boosts, the output voltage signal, for example, via the gain circuitry. The ADC can convert the continuous analog voltage signal into a discrete digital sequence of data samples, or voltage counts. In some embodiments, the ADC can sample the analog voltage signal once every two milliseconds; however, other sampling rates are possible. The ADC can comprise an ADC with 12-bit resolution, which provides 4096 distinct voltage counts.

In some embodiments, the electronic control unit 120 can generate a stable data point to be converted to an angle measurement. The electronic control unit 120 can apply a median filter to the sampled data to eliminate outliers in the data. The output of the median filter can then be fed into a rolling average filter. The rolling average filter can be used to stabilize the data that is converted to an angle measurement. The electronic control unit 120 can implement one or more steps using a finite impulse response (“FIR”) or an infinite impulse response (“IIR”) filter.

In some embodiments, the electronic control unit 120 can convert the voltage count data to an angle measurement in degrees. In performing the conversion, the electronic control unit 120 can be configured to apply a calibration conversion algorithm. The calibration conversion can be configured to account for unit-to-unit variations in components and sensor placement. The calibration routine can be performed for each axis being monitored by the one or more sensors 122. The calibration conversion can comprise removing any mechanical or electrical offsets and applying an appropriate gain calibration for a positive or negative tilt. In some embodiments, the electronic control unit 120 can send the angle measurement to the external output device 130. In some embodiments, the electronic control unit 120 sends raw data from the one or more sensors 122 to the external output device 130. In some embodiments, the electronic control unit 120 sends analog voltage output signals from the one or more sensors 122 to the external output device 130.

In some embodiments, the electronic control unit 120 can include a processor configured to perform one or more of the following functions. The electronic control unit 120 can gather measurements from one or more sensors 122 of the one or more modules 100. The electronic control unit 120 can compile or aggregate data. The electronic control unit 120 can perform calculations to convert the measurements from the one or more sensors 122 to kinematic information. The kinematic information can relate to pelvic mobility. The kinematic information can relate to patient positioning. The kinematic information can be gathered during various motions of the patient such as sitting, leaning, or standing. The module 100 can track pelvic orientation pre-operatively when the patient is sitting. The module 100 can track dynamic pelvic orientation and static pelvic orientation.

The external output device 130 can receive digital signals. As described herein, the one or more accelerometers can output digital signals over a standard digital interface such as I2C and SPI, among others. The external output device 130 can receive digital output signals from the accelerometers. In some embodiments, the external output device 130 only receives signals. In some embodiments, the external output device 130 can send and receive signals. The external output device 130 can include one or more computers, tablets, and/or smartphones. The external output device 130 can include any capabilities of the electronic control unit 120 described herein. The external output device 130 can perform any of the functions of the electronic control unit 120. In some embodiments, the external output device 130 can convert an analog voltage output signal of one or more sensors 122 into an angle degree measurement for presentation on the external output device 130. In some embodiments, the external output device 130 can generate a stable data point to be converted to an angle measurement. In some embodiments, the external output device 130 can convert the voltage count data to an angle measurement in degrees.

In some embodiments, the external output device 130 can send kinematic data. In some embodiments, the external output device 130 can receive kinematic data. In some embodiments, the external output device 130 can send or receive algorithm data. The external output device 130 can communicate with external memory 128 to retrieve and/or store data. The external output device 130 can communicate with external memory 128 to retrieve program instructions for software and/or hardware. The external memory 128 can include memory for the storage of information. The external memory 128 can be a server. The external output device 130 can communicate with the server. The external output device 130 has the capability to send and/or receive algorithm and kinematic data to the server.

The external output device 130 can display data from the module 100. The external output device 130 can display pelvic mobility data. The external output device 130 can display the position or orientation of a portion of the patient's anatomy. The external output device 130 can display range of motion data. The external output device 130 can display data from one or more modules 100. The external output device 130 can display data in real time. The external output device 130 can control operation of the module 100. The external output device 130 can turn on the module 100. The external output device 130 can determine when to collect data from the module 100, such as starting and stopping data collection. The external output device 130 can collect and/or store the data in an application or app installed on the external output device 130. The external output device 130 can transmits data to one or more additional devices such as the external memory 128. The external output device 130 can comprise a light indicator. The light indicator can provide classification information regarding the patient. The light indicator can be a red light and green light. The green light can indicate that the patient has normal mobility. The red light can indicate that the patient has abnormal mobility. The green light can indicate that the population average target angles are appropriate for the patient. The red light can indicate that population average target angles are not appropriate for the patient. The data from the module 100 can have inherent value regardless of whether a surgical method is performed. The module 100 can provide data commensurate with mobility, providing a landscape of kinematic data about the patient. The module 100 can provide data to the cloud. The data can be stored. The data can be compared with earlier or later data. The patient can be tracked over time. The module 100 can provide data for inter-operative use.

The external output device 130 can compare the kinematic information to target or population average kinematic information. Values based on the output of the one or more sensors 122 indicative of kinematics can be compared with population average kinematic data. These population average kinematic data may be based on published averages for healthy patients. The external output device 130 can transmit a patient specific output corresponding to the kinematic information. The patient specific output can be a recommendation to the surgeon. The patient specific output can be a recommendation for an adjustment of the anteversion angle. The patient specific output can be a recommendation for an adjustment of the abduction angle. The patient specific output can be a set of recommended patient specific target angles. The patient specific output can be a recommended adjustment to population average target angles. The patient specific output can be a recommendation to increase or decrease anteversion angle. The patient specific output can be a recommendation to utilize a specific type of implant, such as a dual mobility implant. The patient specific output can be raw data to be analyzed and utilized by the surgeon. The patient specific output can be a graph comparing the patient to the population averages. The patient specific output can take any form usable by the surgeon.

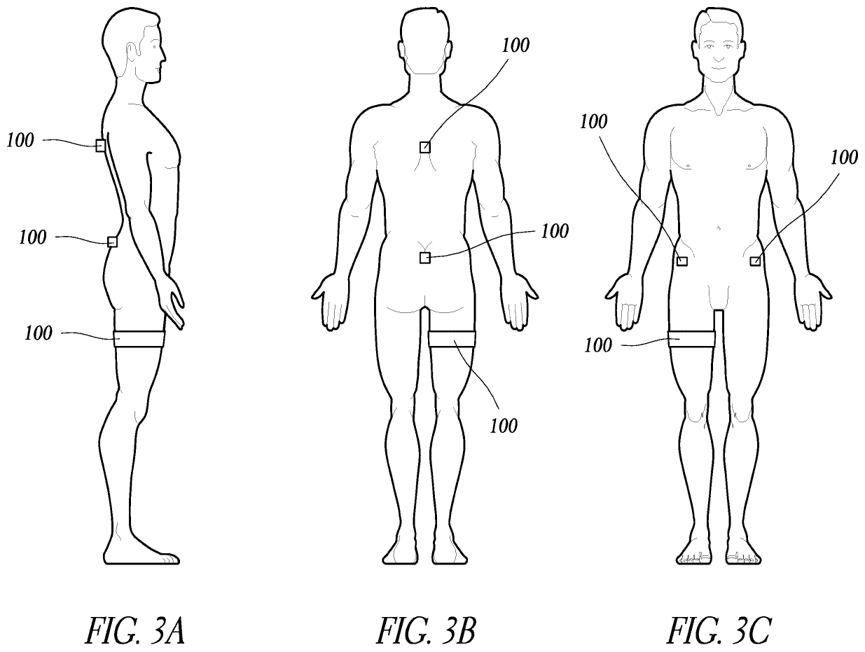

FIGS. 3A-3C illustrate perspective views of the module 100 coupled to a patient. FIGS. 3A-3B illustrate the placement of the module 100 on the sacrum, the module 100 on the spine, and the modules 100 on the ASIS points. In some embodiments, on the sacrum includes a location on a bony prominence that can be identified by palpation of the skin over the sacrum. In some embodiments, the module is positioned on the skin of the patient over the underlying anatomy. The module 100 can be positioned adjacent, near, on, or over any anatomical reference including the ASIS points, pubis. In some embodiments, the mobility assessment can include a pre-operative activity. In some embodiments, positioning the module 100 does not include an incision to position the module 100. Other combinations and positions of the module 100 are contemplated. FIGS. 3A-3C shows the placement of the module 100 on the femur, for instance in a band around the femur. In some embodiments, the module 100 is coupled to a garment configured to be worn by the patient. One or more modules 100 are placed on various parts of the body, such as the sacrum, the femur, and other locations such as the ASIS points, the spine, or other palpable locations on the body. In some methods, one or more modules 100 are placed on the lower back, the upper back, and the neck. The module 100 can be used in an office setting during a pre-operative visit, as described herein.

In some methods, the module 100 is placed by a physician. Movements of the patient can be tracked in a physician's office. The patient can move in a prescribed manner under the direction and supervision of the physician. As an example, the patient may be asked to sit and stand. The patient may be asked to do repetitive motions. The patient may move through a range of motion. The patient may be asked to hold a particular pose or position. The module 100 can record movement over a period of time. The module 100 can record movement when instructed, for instance by the use of a start/stop interface.

In some methods, the module 100 can be used in a home setting. In some methods, the use in an office setting or a home setting is determined by a physician. Movements of the patient can be tracked at the home of the patient. The patient can move freely during normal activities. As an example, the patient may sit, stand, and lean to various degrees during the patient's activities. The patient may move though a range of motion. The patient may repeat movements, such as repeatedly sitting. The patient may move through a range of motion. The patient may be hold a pose or position during normal activities. The module 100 can record movement over a period of time. The module 100 can record movement sporadically over a day, week, or month. The module 100 can record movement once movement is detected. The module 100 can record movement when instructed, for instance by the use of a start/stop interface.

The external output device 130 can receive signals from the module 100 in the home setting. As described herein, the one or more accelerometers can output signals over a standard digital interface. The external output device 130 can include one or more computers, tablets, and/or smartphones of the patient. In some embodiments, the patient interacts with an app on a smartphone or tablet.

The patient's smartphone or tablet can be considered as the external output device 130 or one of the external output devices 130 in the system. The patient may interact with the external output device 130 to identify activities being performed, for instance sitting or standing. The patient may interact with the external output device 130 to identify activities during a timespan. The patient may interact with the external output device 130 to turn on or turn off the module 100. The patient may interact with the external output device 130 to set the time period for recording movement. The external output device 130 can include instructions or prompts for the patient. The external output device 130 can provide instructions on the type of movement needed to be collected.

The patient's device can transmit information to the physician for pre-operative care. The patient's device can transmit information to the surgeon for operative care. The physician can have an external output device 130 to retrieve the information from the module 100. The surgeon can have an external output device 130 to retrieve the information from the module 100. The one or more external output device 130 can transmit or receive information to any component described herein. The one or more external output device 130 can transmit or receive information to a server.

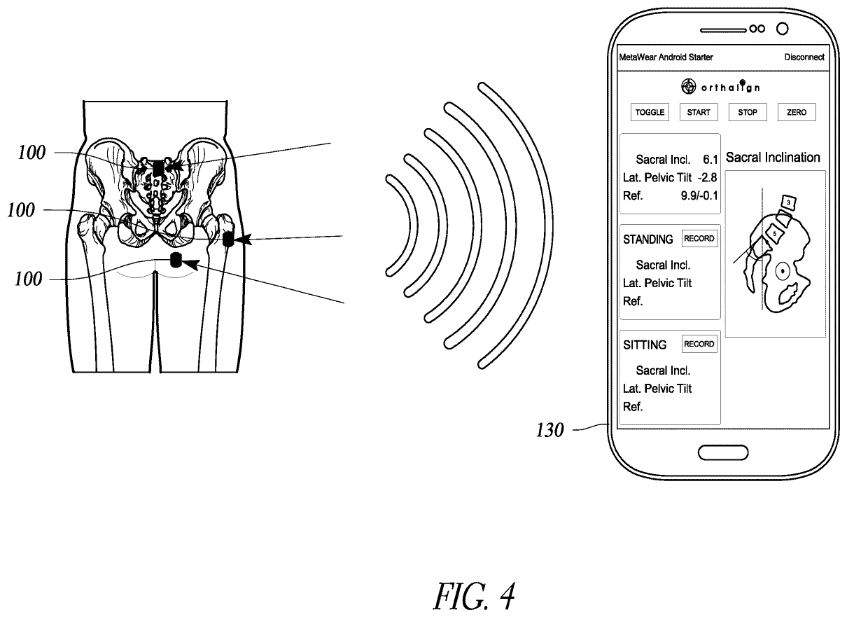

FIG. 4 illustrates view of one or more modules 100 in communication with the external output device 130. The one or more modules 100 can be placed on the patient. The one or more modules 100 can be in communication with the external output device 130. The one or more sensors 122 can provide dynamic data to the external output device 130, directly or indirectly through the electronic control unit 120. The external output device 130 can display one or more values related to pelvic mobility. The external output device 130 can display one or more values related to the position or orientation of the patient's anatomy. The external output device 130 can display the sacral inclination. The external output device 130 can display lateral pelvic tilt. The external output device 130 can display the values related to reference inclination. The external output device 130 can display a graphical representation of pelvic mobility.

The external output device 130 can include any graphical interface. The external output device 130 can include a touchscreen. The external output device 130 can include a display. The external output device 130 can include a screen. The external output device 130 can include one or more digital buttons. The external output device 130 can include one or more icons. The external output device 130 can include a graphical representations of the anatomy. The external output device 130 can include a graphical representation of the spine. The external output device 130 can include a graphical representation of the pelvis. The external output device 130 can include a graphical representation of the upper leg. The external output device 130 can include a graphical representation of the lower back. The external output device 130 can include a graphical representation of any portion of the anatomy. The external output device 130 can include a graphical representation of the location of the modules 100. The external output device 130 can include a graphical representation of one or more anatomical angles. The external output device 130 can include a graphical representation of one or more anatomical axes. The external output device 130 can include a user interface to start measurements such as a touchable button or icon. The external output device 130 can include a user interface to stop measurements such as a touchable button or icon. The external output device 130 can include a user interface to zero measurements of the one or more modules 100. The external output device 130 can include a user interface to change menus or toggle between menus. The external output device 130 can include display of one or more measurements. The external output device 130 can include display of standing measurements. The external output device 130 can include display of sitting measurements. The external output device 130 can include display of real-time measurements.

The external output device 130 can be configured to display one or more on-screen graphics. The on-screen graphics can comprise graphical images or icons. The external output device 130 can be configured to display instructive images, such as illustrated surgical procedure steps. The external output device 130 can be configured to display visual indicators of the orientation information received from the module 100. The external output device 130 can be configured to display degrees of rotation of the module 100 relative to one or more planes. The external output device 130 can be configured to display either a positive or negative sign to indicate direction of rotation from a reference plane. The external output device 130 can be configured to display an interactive indicator to aid a user in maintaining a particular orientation of the module 100. The external output device 130 can be configured to display alphanumeric text or symbols. The external output device 130 can be configured to display directional arrows. While FIG. 4 illustrates an example of the graphical user interface of the external output device 130, other graphical user interfaces are contemplated.

The external output device 130 can display a recommendation to the surgeon. The recommendation can include raw kinematic data from the patient. The recommendation can include a list of characteristics of the patient. The recommendation can include a recommendation to alter target angles to patient specific angles.

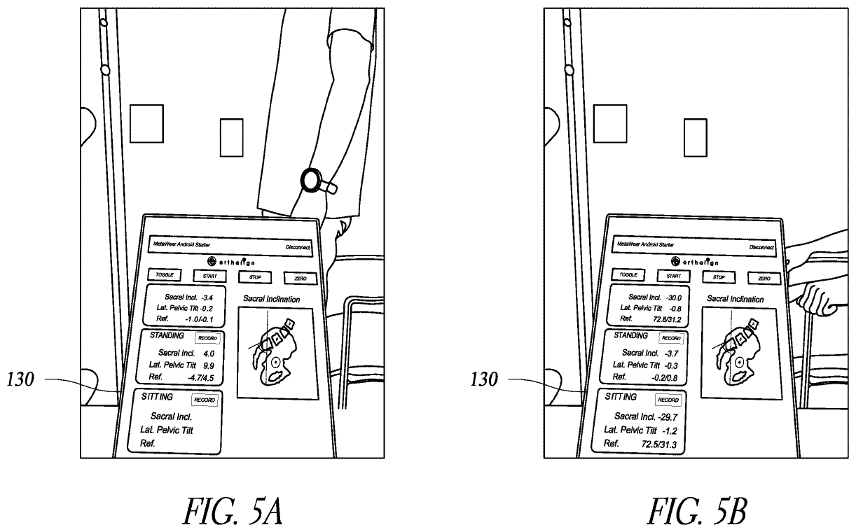

FIGS. 5A-5B illustrate pre-operative methods. Patient mobility using the module 100 can be determined based on a wide variety of movements. The user moves through a range of motion such as standing, sitting, and leaning forward. In some methods, data is captured and processed to determine pelvic mobility and/or other kinematic data. Based on the individualized data from the patient, the system 10 can provide recommendations to the surgeon. In some methods, the system 10 can provide a recommendation to adjust one or more of the target abduction and anteversion angles for total hip arthroplasty. In some methods, the system 10 can provide a recommendation whether to perform a surgical procedure. In some methods, the system 10 can provide a recommendation for the selection of the implant.

The table below provides examples of the guidance provided to the surgeon. The guidance can facilitate the surgeon's choice of target angles. Based on the provided data, the surgeon can alter the anteversion angle and/or abduction angle. In some methods, the patient can move between sitting and standing. The system 10 can compare the sitting and standing characteristics of the patient. The system 10 can compare the patient's measured standing anteversion, sitting anteversion, and change in functional anteversion to population based averages. In some embodiments, the system 10 adjusts the population based averages and outputs patient specific target angles as a recommendation to the surgeon.

In some embodiments, the system 10 provides a recommendation for adjusting the angles based on a comparison of the sitting and standing characteristics of the patient. The system 10 can provide a recommendation, such as decrease anteversion, increase anteversion, or no change in anterversion from the population averages. These recommendations can be displayed on the external output device 130. These recommendations can be sent to the external memory 128 such as a server. These recommendations can be stored for use during a surgical procedure. These recommendations can be utilized when developing a surgical plan.

| | | Change in | |
|---|---|---|---|
| Standing | Sitting | Functional | |
| Anteversion | Anteversion | Anteversion | Recommendation |
| Greater than | Appropriate | Appropriate | Decrease |
| average | | | anteversion |
| Greater than | Less than | Appropriate | No changes in |
| average | average | | anteversion angle |
| Appropriate | Less than | Appropriate | Increase |
| | average | | anteversion |
| Appropriate | Less than | Less than | Increase |
| | average | average | anteversion |
| Appropriate | Appropriate | Less than | Increase |
| | | average | anteversion |
| Greater than | Appropriate | Less than | Decrease |
| average | | average | anteversion |
| Greater than | Less than | Less than | No changes in |
| average | average | average | anteversion angle |
| Appropriate | Appropriate | Appropriate | No changes in |
| | | | anteversion angle |

In some methods, the system 10 including the module 100 and the external output device 130 can produce an output indicative of a standing anteversion of a patient based on measurements from the module 100. For instance, the external output device can display a standing anterversion angle of 3 degrees. This measurements can suggest a neutral standing pelvis. The system 10 can provide an indication of the patient specific standing data. The system 10 including the module 100 and the external output device 130 can produce an output indicative of a sitting anteversion of the patient based on measurements from the module 100. For instance, the external output device can display a sitting anterversion angle of −17 degrees. This measurements can suggest a sitting orientation with sufficient clearance to avoid impingement. The system 10 can provide an indication of the patient specific sitting data. The system 10 including the module 100 and the external output device 130 can produce an output indicative of patient mobility. For instance, the external output device can display a mobility indicative of 20 degrees range of motion. This measurements can suggest a standard range of motion. The system 10 can provide an indication of the patient specific mobility data. One or more of these outputs can be utilized by the surgeon to determine the patient specific target anteversion for optimal stability.

For another patient, the system 10 including the module 100 and the external output device 130 can output a standing anteversion of −1 degrees. This measurements can suggest a neutral standing pelvis. The system 10 including the module 100 and the external output device 130 can output a sitting anteversion of −10 degrees. This measurements can suggest a sitting orientation with insufficient clearance to avoid impingement. The system 10 including the module 100 and the external output device 130 can output a mobility of 9 degrees range of motion. This measurements can suggest a decreased range of motion. One or more of these outputs can be utilized by the surgeon to determine whether to increase target anteversion to avoid impingement and improve posterior stability when sitting.

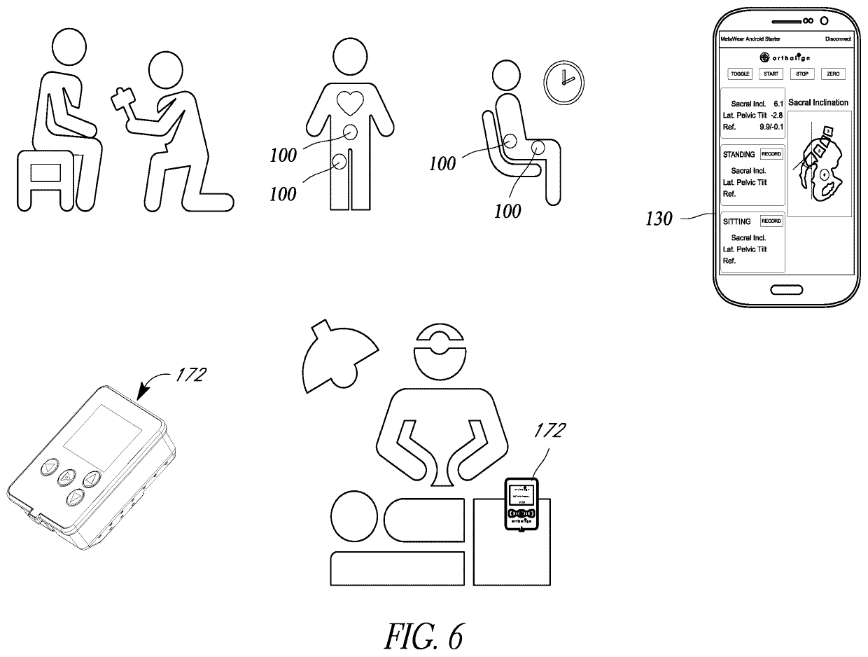

FIG. 6 illustrates an embodiment of a surgical workflow. The module 100 can be incorporated into a pre-operative workflow. The pre-operative workflow can be separated in time and space from an operative workflow. The module 100 can be easily integrated into the current workflow. The module 100 can facilitate pre-operative, objective planning, as described herein.

In some methods of use, the patient can have a preoperative visit as shown in FIG. 6. The one or more modules 100 can be mounted to the body of the patient. In some methods of use, the module 100 is placed on the sacrum. In some methods of use, at least one module 100 must be placed on the sacrum. In some methods of use, the module 100 is placed on the coccyx. In some methods of use, at least one module 100 must be placed on the coccyx. In some methods of use, the module 100 is placed on the ilium. In some methods of use, the module 100 is placed on iliac crest. In some methods of use, the module 100 is placed on iliac spine. In some methods of use, the module 100 is placed on the acetabulum rim. In some methods of use, the module 100 is placed on the ischium. In some methods of use, the module 100 is placed on the pubis. In some methods of use, the module 100 is placed on the pubis tubercle. In some methods of use, the module 100 is placed on the pubis symphysis. In some methods of use, at least one module 100 must be placed on the pelvis.

In some methods, the module 100 can be placed on one of the ASIS points. The module 100 on the sacrum can be used in combination with the module 100 on one of the ASIS points. In some methods, modules 100 are placed on both ASIS points. The module 100 on the sacrum can be used in combination with the modules 100 on both of the ASIS points. In some methods, the module 100 can be placed on one of the PSIS points. The module 100 on the sacrum can be used in combination with the module 100 on one of the PSIS points. In some methods, modules 100 are placed on both PSIS points. The module 100 on the sacrum can be used in combination with the module 100 on both of the PSIS points.

In some methods, the module 100 can be placed on one of the femurs. The module 100 on the sacrum can be used in combination with the module 100 on one of the femurs. In some methods, modules 100 are placed on both femurs. The module 100 on the sacrum can be used in combination with the modules 100 on both of the femurs. In some methods, modules 100 are placed on one or both tibias. In some methods, modules 100 are placed on one or both patella. In some methods, modules 100 are placed on one or both fibula. In some methods, modules 100 are placed on one or both feet. In some methods, modules 100 are placed on one or both tarsus. In some methods, modules 100 are placed on one or both calcaneus. In some methods, modules 100 are placed on one or both metatarsals. In some methods, modules 100 are placed on regions on one or both legs. The module 100 on the sacrum can be used in combination with the modules 100 on one or both legs.

In some methods, the module 100 is placed on the spine. The module 100 on the sacrum can be used in conjunction with the module 100 on the spine. In some methods, the module 100 is placed on the lower back. The module 100 on the sacrum can be used in conjunction with the module 100 on the lower back. In some methods, the module 100 is placed on the upper back. The module 100 on the sacrum can be used in conjunction with the module 100 on the upper back. In some methods, the module 100 is placed on the neck. The module 100 on the sacrum can be used in conjunction with the module 100 on the neck. In some methods, the module 100 is placed on one or more bones of the chest. In some methods, the module 100 is placed on one or more ribs. In some methods, the module 100 is placed on a portion of the ribcage. In some methods, the module 100 is placed on the sternum. The module 100 on the sacrum can be used in combination with the modules 100 on the sternum. In some methods, the module 100 is placed on the clavicle or collarbone. In some methods, the module 100 is placed on one or more vertebrae (e.g., cervical vertebrae, thoracic vertebrae, lumbar vertebrae, sacral vertebrae, coccygeal vertebrae, and any combination of vertebrae). The module 100 on the sacrum can be used in combination with the modules 100 on one or more vertebra.

In some methods, modules 100 are placed on any two locations on the body. In some methods, a module 100 is placed on one or more locations on the body (e.g., one location, two locations, three locations, four locations, five locations, six location, seven locations, eight locations, nine locations, ten locations or any range of the foregoing values.). In some methods, modules 100 are placed on one or more contralateral locations on the body. In some methods, modules 100 are placed on one or more symmetrical locations. In some methods involving two or more modules 100, the two or more modules 100 can be used in combination. In some methods involving two or more modules 100, the two or more modules 100 can collect measurements simultaneously. In some methods involving two or more modules 100, the two or more modules 100 can collect measurements independently. In some methods, modules 100 are positioned on a variety of portions of the body.

In some methods, the module 100 can be placed on one or more palpable locations. In some methods, the module 100 can be placed on the cerivo-thoracic junction. In some methods, the module 100 can be placed on any surface of the skin of the patient. In some methods, the module 100 can be placed on any surface of the skin of the patient overlying bone. The module 100 can be coupled to at least one anatomical region. In some methods of use, the module 100 is coupled to at least one anatomical region with little soft tissue. The soft tissue may move relative to the underlying bone. The module 100 can be configured to track the underlying bone.

The one or more modules 100 can output data when the patient is standing. The one or more modules 100 can output data when the patient is sitting. The one or more modules 100 can output data when the patient is any position or during any movement. In some embodiments, the module 100 comprises one or more sensors 122 measuring position, orientation, and/or movement. The data from the one or more sensors 122 can be captured. The data from the one or more sensors 122 can be processed. The standing data can relate to the sitting data. The range of motion data can be compiled. The range of motion data can relate to the patient specific target angles. In some methods, the patient specific target angles can be part of the pre-operative plan to determine patient-specific target angles.

In some embodiments, one or more modules 100 are utilized. The one or more modules 100 can measure pelvic mobility. This pelvic mobility measurement can be used in surgical planning. The one or more modules 100 can collect dynamic mobility measurements. The patient can move in a variety of ways, such as standing, sitting, leaning, lifting one leg, lifting the other leg, squatting, or other motions. The one or more modules 100 can collect dynamic mobility measurements during movement by the patient. The one or more modules 100 can collect dynamic mobility along the movement trajectory from sitting to standing. The one or more modules 100 can send data to the external output device 130. The module 100 can interact with the external output device 130, such as a smartphone with an app. These steps can be completed in the office or home setting. These steps can be completed pre-operatively.

In some embodiments, surgeon can review data from the module 100. In some embodiments, surgeon can review data from the module 100 on the external output device 130. In some embodiments, the external output device 130 can communicate with the external memory 128. In some embodiments, surgeon can review data from the module 100 on the external memory 128. In some embodiments, surgeon can review data from the module 100 on the external output device 130 that retrieves information from the external memory. In some embodiments, the external output device 130 and/or the external memory 128 can communicate with one or more operative devices. In some embodiments, surgeon can review data from the module 100 on a surgical orientation device 172. The surgical orientation device 172 can be used during a surgical operation, such as total hip arthroplasty. In some embodiments, the surgical orientation device 172 can receive patient specific data obtained from the one or more modules 100.

The surgeon can determine target cup angles based on, at least in part, the data from one or more modules 100. The surgeon can utilize his judgement in interpretation of the patient specific data. The surgeon can accept a recommendation from the system 10. The surgeon can reject a recommendation from the system 10. The surgeon can modify a recommendation from the system 10. Once the target angles are determined by the surgeon, the target angles can be an input for the surgical orientation device 172. The surgical orientation device 172 can facilitate the navigation of the cup to the target angles.

In some embodiments, the patient specific target angles can be displayed on the surgical orientation device 172 for use in the operating room. In some embodiments, the module 100, and data from the module 100, can be incorporated without changing current surgical workflow.

In some embodiments, the module 100 is utilized in an operative setting. The module 100 can provide data related to patient positioning. The module 100 can determine if the hip is correctly placed. The module 100 can be placed on an anatomical location. Two or more modules 100 can be placed on anatomical locations. In some methods, the module 100 can determine if the ASIS line is horizontal. In some methods, the module 100 can determine if the ASIS line is vertical. In some methods, the module 100 can determine if the pelvis is vertical. In some methods, the module 100 can determine if the pelvis is horizontal. In some methods, the module 100 can determine if the patient is properly positioned for an anterior approach. In some methods, the module 100 can determine if the patient is properly positioned for a posterior approach.

In some embodiments, the module 100 comprises one or more sensors 122 measuring position, orientation, and/or movement during a surgical procedure. The data from the one or more sensors 122 can be captured. The data from the one or more sensors 122 can be processed. The patient can be adjusted to achieve the proper positioning. The patient's anatomy can be tracked during the surgical procedure. The module 100 can track pelvic rotation. The module 100 can track pelvic orientation. The module 100 can track the alignment of the anatomy during a surgical procedure. The module 100 can ensure that the ASIS line is aligned with vertical or horizontal, depending on the surgical approach.

The module 100 can provide several advantages. The module 100 can allow the surgeon to individualize and adjust the pre-operative plan. The module 100 can allow the surgeon to improve the pre-operative plan based on patient specific pelvic mobility. The module 100 can facilitate individualized cup placement. Individualized cup placement target angles can be more effective and lead to better clinical outcomes. The module 100 can take into account patient specific pelvic tilt. The module 100 can account for the patient specific range of motion. The surgeon can consider patient specific activities for daily living when determining the patient specific target angles. The surgeon can consider changes from supine or lateral to weight bearing or standing when determining the patient specific target angles. The surgeon can consider adjusting to match functional anteversion when determining the patient specific target angles.

The module 100 can be used to provide kinematic or other patient specific data to the surgeon. The surgeon can use this data to determine patient specific target angles. The module 100 can be used to modify the target anteversion angle. The module 100 can be used to determine patient-specific anteversion angles. The module 100 can be used to determine a target functional anteversion. In some embodiments, the module 100 can advantageously prevent or reduce instability. Another advantage is that the module 100 can prevent or reduce dislocations. Yet another advantage is that the module 100 can prevent or reduce impingements. The guidelines for cup placement can include the General (Lewinnek) Safe Zone. This safe zone can be sub-optimal for cup placement guidance. The module 100 can improve upon this safe zone by providing recommendations to adjust the target angles based on pre-operative measurements. The surgeon can accept, reject, or modify any recommendation by the system 10.

The module 100 can be advantageously used to classify patients. Pelvic tilt is relevant to total hip arthroplasty. There is a need to identify patients that are good candidates for typical total hip arthroplasty, as well as identify patients that are good candidates for various implant types, such as dual mobility. Pelvic tilt is relevant to determining whether a target cup angle should be adjusted. Patient anatomy can have a variety of deformities impacting pelvic tilt. The patient can have a spinal deformity, such as a sagittal spinal deformity, that influences pelvic tilt. Previous spinal surgeries such as fusion can impact pelvic mobility. Dislocations rates are higher for patients with previous spinal surgery. The alignment between the spine and the pelvis can impact the target anteversion angle. There is a need to determine patient specific target angles to improve clinical outcomes.

The module 100 can provide information on characteristics of the patient. These characteristic can be used to classify patients. These characteristic can be used to modify a surgical plan, for instance by determining whether to alter population average target angles. These characteristics can be used to determine if a patient is a candidate for surgery. These characteristics can be used to determine what type of surgery to perform.

These characteristics can be used to determine what type of implant to use. The module 100 can provide information on whether to use a dual mobility implant. In some uses, dual mobility hip replacements reduce the risk of dislocations for certain patients. The module 100 can provide information useful in determining pre-operatively which patients would benefit from a dual mobility implant. Dual mobility implants comprise a femoral head which is captive but mobile within a liner, wherein the liner is captive but mobile within an acetabular shell. The module 100 can determine mobility characteristics of the patient, which can be used by the surgeon to select the implant. The module 100 can determine whether the patient has abnormal mobility or normal mobility. These characteristics can be used to determine whether to use a patient specific implant. The information from the module 100 can help to assess the risk of surgery, such as the risk of dislocation. These characteristics can be used to determine what type of jig or surgical instrumentation to use. These characteristics can be used to determine whether to use a patient specific jig.

In some embodiments, the module 100 can be used in combination with x-rays or other imaging techniques. In some methods of use, the patient undergoes x-ray assessments. The x-ray assessments can provide snapshots of the pelvic range of motion using a variety of positions. An x-ray can be taken while the patient is standing, while the patient is sitting, and/or while the patient is leaning. In some methods, the x-ray assessments can have limited effectiveness. These assessments include cumbersome, manual processes to position the patient. The x-ray assessments may require large and expensive equipment, with limited access. The x-rays or other imaging technique also pose a risk to patient by increasing exposure to radiation. These assessments merely provide snapshot or static image of the patient. In some methods, these images provide less useful information than dynamic measurements. The module 100 can improve upon pre-operative x-ray data because the sensor data is dynamic, over a continuous range of motion.

In some embodiments, the module 100 can be used instead of imaging techniques such as x-rays. The module 100 can provide dynamic measurements of pelvic mobility. The module 100 can provide measurements of patient positioning. In some embodiments, the target angles are determined without the need for pre-operative x-rays related to patient mobility. Rather, the surgeon can rely on the range of motion detected by the module 100 to determine patient specific target angles.

The module 100 can have a broad purpose to determine patient characteristics. The module 100 can determine mobility. The module 100 can determine patient positioning. The module 100 can provide static or dynamic information that is specific to the patient being monitored. The module 100 can have a narrower purpose to provide an assessment of the patient to the surgeon. The assessment can relate to patient specific mobility. The assessment can relate to whether the patient has normal or abnormal mobility. The assessment can relate to whether the patient is a good candidate for a specific type of surgery or implant. The assessment can provide additional or alternative data from traditional imaging techniques. With this data, the surgeon can determine the surgical plan such as determining patient specific target angles. The module 100 is a useful tool to provide data to the surgeon.

The module 100 can be used in combination with intra-operative navigation. The module 100 can improve precision and accuracy of intra-operative navigation. In some embodiments, the module 100 can be used for intelligent targeting of an individualized anteversion angle. The module 100 can provide pre-operative data to the surgeon to better predict target abduction and anteversion angles for cup placement. The goal is to improve patient outcomes that can be impacted by cup placement and targeting.

The module 100 can be used for objective surgical planning. The module 100 can be used for tracking weight loss pre-operatively. The module 100 can be used for tracking muscle tone pre-operatively. The module 100 can be used to determine for readiness for surgery. The module 100 can be used for user compliance with a pre-operative program. The module 100 can be used for communication. The module 100 can be used for smart, individualized, economical, and connected care. The module 100 can be used to reduce costs and complications.

### B. Hip Replacement Systems for Anterior and Poster Approach

### 1. Hip Navigation System

A variety of systems and methods are discussed below that can be used to improve outcomes for patients by increasing the likelihood of proper placement of a medical prosthesis. These systems can be focused on inertial navigation techniques to establish a reference plane. These systems can be focused on inertial navigation techniques to guide a medical prosthesis relative to the reference plane. In some methods, any intra-operative navigation system can be utilized. The intra-operative navigation system can navigate to determined target cup angles. In some methods, the target cup angles can be determined, at least in part, on data from the one or more modules 100. The methods can utilize one or more steps described below. In some methods, the modules 100 are utilized for other purposes than for use with navigation.

The systems and methods can include a primary hand-held electronic surgical orientation device with a user interface and a set of inertial sensors, a secondary electronic orientation device with a second set of inertial sensors and a jig to couple the orientation devices to the pelvis. The electronic orientation devices can establish a reference plane before the cup is placed. The secondary electronic orientation device can be coupled to a cup inserter to monitor the abduction and anteversion angles relative to this reference plane during cup placement. Features of the system are described herein.

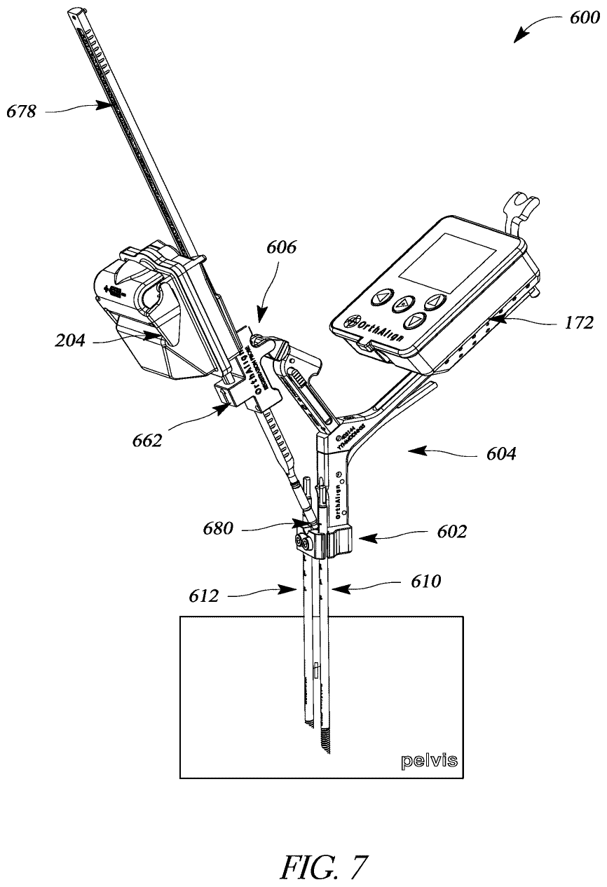

FIG. 7 shows a hip navigation system 600 adapted to navigate a hip joint procedure with reference to anatomical landmarks. The system 600 is shown mounted on a pelvis in FIG. 7, which is shown as a box for simplicity. The system 600 can be mounted on a pelvis for a posterior approach as described herein. The system 600 can be mounted on a pelvis for an anterior approach as described herein.

The system 600 can include a fixation base 602, a first assembly 604, and a second assembly 606. The first assembly 604 is rigidly connected to the hip or pelvis in the illustrated configuration so that motion of the pelvis causes corresponding motion of sensor(s) in the first assembly 604 as discussed herein. Sensing this motion enables the system 600 to eliminate movement of the patient as a source of error in the navigation. The second assembly 606 provides a full range of controlled motion and sensor(s) that are able to track the motion, in concert with sensor(s) in the first assembly 604.

The sensors in assemblies 604, 606 preferably transfer data among themselves and in some cases with external devices including the module 100, the external output device 130 such as computers, tablets, and/or smartphones, and external displays. The assemblies 604, 606, the module 100, and the external output device 130 can transfer data wirelessly, using Bluetooth, Wifi® or other standard wireless telemetry protocol. The system 600 can include one or more fixation pins 610, 612. The system 600 can also include a surgical orientation device 172 and an orientation sensing device 204, as described herein. The patient specific target angles determined by the surgeon can be an input into the surgical orientation device 172. The surgical orientation device 172 and the orientation sensing device 204 can facilitate navigation of an acetabular cup to the desired target angle.

The surgical orientation device 172 detects orientation and rotation of the device 172 relative to a reference frame. The surgical orientation device 172 preferably comprises at least one sourceless, inertial sensor, such as an accelerometer, a gyroscope, or a combination of these sensors and other sensors. In some embodiments, the surgical orientation device 172 includes a three axis accelerometer to detect orientation relative to gravity and a plurality of gyroscopes to detect rotation. Other sensors could be used in various modifications. Examples of specific sensor combinations include Analog Devices ADIS 16445 and Invensense MPU-6050 or MPU-9150 among others. In some embodiments, the surgical orientation device 172 can be disposable and the sensors can be inexpensive sensors. In some embodiments, the surgical orientation device 172 is disposable. In some embodiments, the surgical orientation device 172 is reusable.

The surgical orientation device 172 can include one or more sensors that together form an inertial measurement unit (IMU). In some embodiments, the IMU can include a first sensor for determining acceleration and a second sensor for determining gyroscopic positioning. As discussed herein, the first sensor can be an accelerometer and the second sensor can be a gyroscopic sensor. In some embodiments, the sensors can comprise a three-axis gyroscopic sensor and a three-axis accelerometer sensor. The surgical orientation device 172 can include a transmitter for sending data or receiving data from one or more sensors of the system 600, such as one or more sensors of the orientation sensing device 204. The surgical orientation device 172 can include a transmitter for sending data or receiving data from the module 100 and the external output device 130. The information received from the orientation sensing device 204 can be fed to an input port, or alternatively, the electronic control unit can itself receive the information (e.g., wirelessly). The information from the orientation sensing device 204 can correspond, for example, to the position and/or orientation of the orientation sensing device 204 and can be used by the surgical orientation device 172 to determine an aggregate, or overall, position and/or orientation of the surgical orientation device 172. The information from the module 100 and/or the external output device 130 can correspond, for example, to pelvic mobility and can be used by the surgical orientation device 172 to navigate to patient specific target angles.

The system 600 can include the second assembly 606. The second assembly 606 can include a dock 662 sized to accept a probe 678 therethorugh. The probe 678 has a distal end 680 designed to touch a point or a location or locations, as described herein. The distal end 680 can be straight as shown. In other embodiments, the distal end 680 is slanted or curved. The probe 678 can be coupled to the dock 662 such that the probe 678 is movable. This distal end 680 of the probe 678 can pivot or rotate to contact anatomical landmarks. The probe 678 can be slid relative to the dock 662 to different translational positions relative to the attachment location of the fixation pins 610, 612. The slideability of the probe 678 within the dock 662 enables the distal end 680 to move to reach a point or anatomical landmarks in the same plane of the probe 678 but closer to or farther from the distal end 680. The second assembly 606 permits a range of motion of a distal end 680 of the probe 678 to facilitate acquiring a point that that is spaced apart from the attachment location of the fixation pins 610, 612. The second assembly 606 permits a range of motion of a distal end 680 of the probe 678 to facilitate acquiring a plurality of landmarks that are different distances from the attachment location of the fixation pins 610, 612. While the probe 678 is illustrated as an elongate member, other configurations are contemplated. Any indicator can be utilized to establish an orientation or position of a point. In some embodiments, a light or laser can be an indicator to establish position and/or orientation of a point. In some embodiments, a camera can be an indicator to establish position and/or orientation of a point. In some embodiments, a probe can be an indicator to establish position and/or orientation of a point. In some embodiments, a probe with one or more scales or codes can be an indicator to establish position and/or orientation of a point. In some embodiments, a probe with a computer readable codes such as a QR code can be an indicator to establish position and/or orientation of a point.

The orientation sensing device 204 detects orientation and rotation of the probe 678, as described herein. The orientation sensing device 204 preferably comprises at least one sourceless sensor, such as an accelerometer, a gyroscope, or a combination of these sensors and other sensors. In some embodiments, the orientation sensing device 204 includes a three axis accelerometer to detect orientation relative to gravity and a plurality of gyroscopes to detect rotation. Other sensors could be used in various modifications. In some embodiments, the orientation sensing device 204 is reusable.

In some methods of use, or steps thereof, the orientation sensing device 204 is coupled to the probe 678 such that movement of the probe 678 corresponds to movement of the orientation sensing device 204. In some methods of use, or steps thereof, the orientation sensing device 204 is coupled to a non-movable, static portion of the second assembly 606 such as for calibration. In some methods of use, or steps thereof, the orientation sensing device 204 is coupled to a movable portion of the second assembly 606 such as the dock 662. In this position, the orientation sensing device 204 can determine a length measure or extension of the probe 678. In some embodiments, the probe 678 can include a marking or a scale. In some embodiments, the orientation sensing device 204 can include a camera. The camera can capture images of the markings of the probe 678.

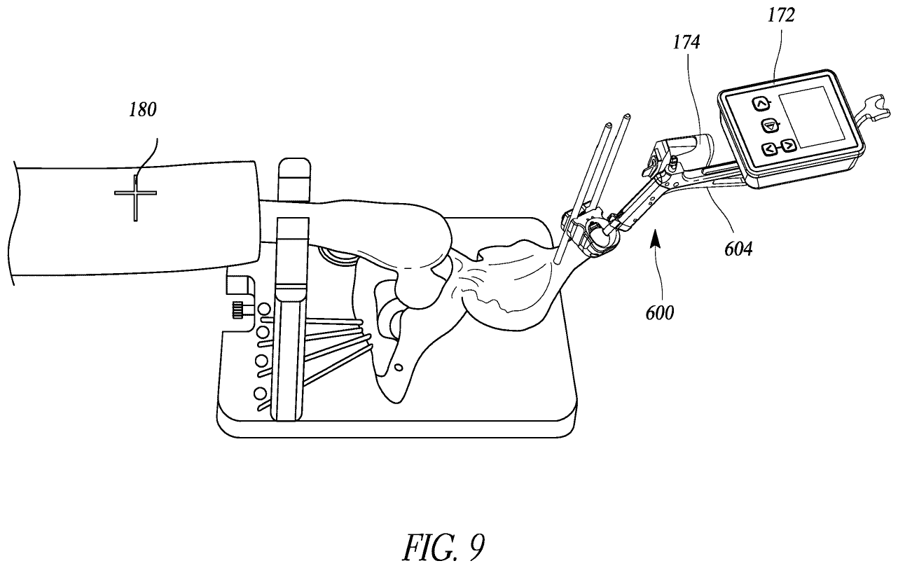

In some methods of use, the system 600 includes an optical component 174 shown in FIG. 9. The optical component 174 can be any device designed to project light including visible, ultraviolet, and infrared light. The optical component 174 can comprise one or more lasers, which can be configured to project laser light. The laser can emit light with a very narrow spectrum, for instance a single color of light. In some embodiments, the optical component 174 is integrated into the surgical orientation device 172. In some embodiments, the optical component 174 can be a separate component from the surgical orientation device 172. The optical component 174 can be located beside or adjacent to or above the surgical orientation device 172. The display of the surgical orientation device 172 can include instructions related to the method of using the optical component 174. In some embodiments, the optical component 174 can be rotated, pivoted, or otherwise moved relative to the first assembly 604. The rigidity of the system 600 can secure the position of the optical component 174 once moved into position.

In some methods of use, the optical component 174 can be positioned and/or moved until the optical component 174 projects light on a selected portion of the anatomy. In some methods of use, the optical component 174 can be positioned and/or moved until the optical component 174 projects light on a target probe, as described herein. In some methods of use, the optical component 174 can be positioned and/or moved until the optical component 174 projects light on sterile wrap, medical drape, bandage, tape, and/or other instruments.

The optical component 174 can emit a pattern of light. Examples of patterns of lights include one or more lines, one or more points, one or more plane, or one or more shapes. The optical component 174 can be moved until the light is projected on at least one anatomical region. In some methods of use, the light is projected onto at least one anatomical region with little soft tissue. The soft tissue may move relative to the underlying bone. The surgeon can select locations to illuminate where the skin is close to the underlying bone. In some methods of use, the optical component 174 can project a pattern onto a portion of the anatomy or a target coupled to the anatomy. The optical component 174 can project one or more points, one or more lines, one or more planes, one or more shapes, one or more colors, and/or one or more patterns. In some embodiments, when the projection of light incident on a surface is visible as two intersecting lines, the pattern may be referred to herein as a cross-hair. The optical component 174 can be used to perform one or more methods or method steps.

### 2. Methods for Posterior Approach

FIG. 7 illustrate hip navigation system 600 which can be adapted to navigate a hip joint procedure from a posterior approach.

In some methods of cup placement in total hip arthroplasty, the inclination and anteversion angles are with respect to the Anterior Pelvic Plane (defined as a plane created by the two anterior superior iliac spines (ASIS) and the pubic symphysis). While these anatomical features are visible/palpable while the patient is in a supine position, the majority of total hip replacements are accomplished via a posterolateral approach with the patient in some variation of a lateral position, in which most of these landmarks are not accessible or visible. Historically, navigation for posterior approach hip replacement has been accomplished by registering the anatomical features of the Anterior Pelvic Plane with the patient first in a supine position and, once this plane is recorded by the navigation computer, moving the patient to a lateral position in order to perform hip surgery—with navigation performed with respect to the directly registered Anterior Pelvic Plane. This approach to hip navigation is sub-optimal for surgical workflow because the extra movement of the patient from supine to lateral position takes more surgeon and staff time and requires breaking sterility and re-draping. This is one of the key reasons why hip navigation has failed to be adopted by most of the market.

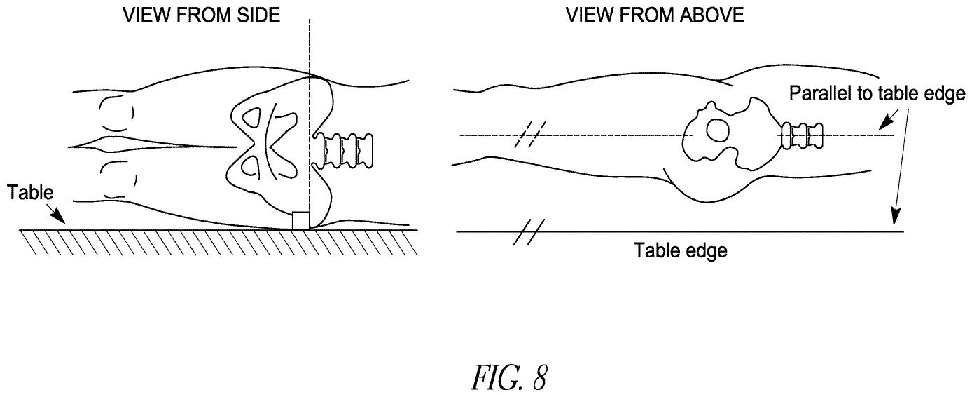

FIG. 8 shows an embodiment of the patient positioning for the posterior hip approach. Most hip replacement procedures presently are performed from a posterior approach. In this approach, the patient is positioned on his/her side and the Anterior Pelvic Plane is oriented vertically, e.g., perpendicular to the plane of the table on which the patient is positioned. In the posterior hip approach, the patient can be placed on their side. In some methods of use, the patient can be placed in the lateral decubitus position. When positioning patient prior to surgery, the surgeon can align the anterior pelvic landmarks (both ASISs and pubic tubercle) in a vertical plane parallel to the long edge of the operating table. In some methods of use, when positioning patient prior to surgery, the surgeon can align the anterior pelvic landmarks (both ASISs and pubic tubercle) in a vertical plane perpendicular to a surface of the table. In some methods of use, when positioning patient prior to surgery, the surgeon can align the anterior pelvic landmarks (both ASISs and pubic tubercle) in a vertical plane parallel to gravity. In some methods of use, when positioning patient prior to surgery, the surgeon can align Anterior Pelvic Plane parallel to gravity. In some methods of use, when positioning patient prior to surgery, the surgeon can align Anterior Pelvic Plane vertically.

In some methods of use, the system 600 calculates cup angles based on the assumption that the pelvis is accurately positioned. In some methods of use, the system 600 calculates cup angles based on the assumption that the Anterior Pelvic Plane is vertical during the posterior procedure. The surgeon can ensure that pelvis is securely held by an appropriate positioning device such as a peg board or vise-type patient positioner. The surgeon can verify that patient is positioned in an appropriate position for the posterior approach. Correct patient positioning is important for accurate navigation.

The system 600 can be partially assembled for calibration. In some embodiments, the first assembly 604 can be assembled. The surgical orientation device 172 can be coupled to the first assembly 604. In some embodiments, the second assembly 606 can be assembled. The orientation sensing device 204 can be coupled to the second assembly 606. The orientation sensing device 204 can be coupled to a non-movable portion of the second assembly 606. The orientation sensing device 204 can be fixed in position relative to the surgical orientation device 172. The angle between the orientation sensing device 204 and the surgical orientation device 172 can be fixed. The surgical orientation device 172 and the orientation sensing device 204 can be calibrated.

The system 600, or a portion thereof, can be attached to the pelvis as shown in FIG. 9. In some methods, the fixation pins 610, 612 are placed in the ipsilateral iliac crest. The surgeon can slide the fixation base 602 over both pins 610, 612. The first assembly 604 can be secured to the fixation base 602. The surgical orientation device 172 can be attached to the first assembly 604 or can be separately coupled once the first assembly 604 is secured. The optical component 174 can be secured to the system 600. In some embodiments, the optical component 174 can be secured to the first assembly 604.

The surgeon can place the leg on the operative side in a neutral position. The surgeon can align the optical component 174 so that the laser projects a pattern of light as shown in FIG. 9. The surgeon can adjust the optical component 174 to project light onto an anatomical landmark or target. The method can include marking the incidence of light 180. The method can include the step of marking two or more points along a line of light. The method can include drawing a line along the line of light. The method can include capturing an image of the incidence of light 180. In some embodiments, the surgeon traces the pattern of light. In some embodiments, the surgeon can make a mark on the femur, such as a bovie mark, a pen mark, a stitch or other durable indication. In some embodiments, the surgeon can mark the incidence of light 180.

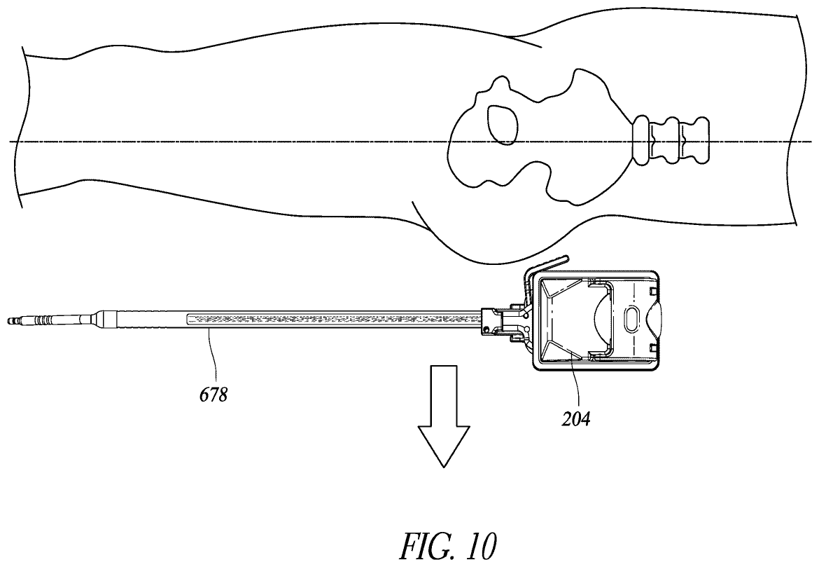

FIG. 10 illustrates one embodiment of registering the table. FIG. 10 is a top view with the patient in the lateral decubitus position of the posterior approach. The surgeon can verify the sagittal plane of pelvis is level. During positioning of the patient, the Anterior Pelvic Plane is positioned vertically. The Anterior Pelvic Plane can be defined as a plane created by the two anterior superior iliac spines (ASIS) and the anterior surface of the pubic symphysis. The Anterior Pelvic Plane can be oriented vertically when the patient is in the lateral decubitus position of the posterior approach. The table plane can be a horizontal or generally horizontal plane.

During table registration, the probe 678 is coupled with the orientation sensing device 204. In some methods of use, the probe 678 and the orientation sensing device 204 are constrained such that movement of the probe 678 causes movement of the orientation sensing device 204. FIG. 10 illustrates one position of the probe 678 to calculate the direction of gravity when the probe 678 is generally horizontal. The probe 678 can be aligned with the long axis of the body. The probe 678 can be held substantially parallel to the plane of the table. In some embodiments, the user can align the probe 678 with the horizontal. The user can visually inspect the probe 678 from one or more locations to verify alignment with the table plane. As described herein, the probe 678 coupled to the orientation sensing device 204 can be positioned horizontally or substantially horizontally to measure gravity. The probe 678 can be held steady during table registration.

FIG. 10 illustrates the probe 678 uncoupled from the second assembly 606. In some embodiments, the probe 678 is uncoupled from the second assembly 606 during registration of the table plane. In other embodiments, the probe 678 is coupled from the second assembly 606 during registration of the table plane. The probe 678 can be coupled to the dock 662 and held in a horizontal position.

In some embodiments, the position and/or orientation of the orientation sensing device 204 can be recorded by the surgical orientation device 172 when the probe 678 is held horizontally. In some embodiments, the gravity measurements are done when the probe 678 is positioned horizontally. The user can position the probe in approximately a horizontal position by aligning the probe with horizontal and/or vertical reference surfaces in the surgical field. The sensors in the orientation sensing device 204 can sense the direction of gravity to provide an estimate of horizontal and/or vertical axes. In some embodiments, the orientation sensing device 204 and the surgical orientation device 172 can detect the direction of gravity by other methods. In some embodiments, the orientation sensing device 204 is held vertical to sense the direction of gravity. In some embodiments, the orientation sensing device 204 is held at any angle relative to horizontal. In some embodiments, the orientation sensing device 204 is held at any angle relative to vertical. The user can enter an input to register the table (e.g., depress a button on the surgical orientation device 172). The user can interact with a user interface on the surgical orientation device 172 to signal to the surgical orientation device 172 to capture data of the orientation sensing device 204. The surgical orientation device 172 can indicate that data was recorded. In some methods of use, the table registration can be taken at any point during the procedure. The table registration can be taken during pre-operative calibration. The table registration can be taken during intra-operative calibration. The surgical orientation device 172 can store the table plane and/or the table reference frame.

The table registration can utilize a measurement of gravity. As described herein, the surgical orientation device 172 comprises one or more inertial sensors. As described herein, the orientation sensing device 204 comprises one or more inertial sensors. In some embodiments, inertial data from one or more inertial sensors is used to calculate the vertical plane and/or horizontal plane. In some embodiments, the position and/or the orientation data of one or more inertial sensors is used to calculate the table plane or table reference frame. The surgical orientation device 172 and/or the orientation sensing device 204 can comprise an accelerometer, which can provide a measurement of the direction of gravity. The accelerometer at rest can measure the acceleration due to Earth's gravity. The accelerometer can measure the acceleration from gravity straight downward or vertically. The accelerometer can produce a vertical vector. The accelerometer can produce a horizontal vector by transforming the vertical vector (e.g., by rotation of 90 degrees). The accelerometer can provide orientation and/or position data such that the table plane is perpendicular to the force of gravity. In some embodiments, the surgical orientation device 172 and/or the orientation sensing device 204 includes a sensor to detect the direction of gravity. In some methods of use, the orientation sensing device 204 and the probe 678 can be positioned in other ways than horizontally to measure the direction of gravity.

The surgical orientation device 172 and/or the orientation sensing device 204 can provide a reference to gravitational zero. Gravitational zero, as referred to herein, refers generally to an orientation in which an axis of a sensor is perpendicular to the force of gravity, and thereby experiences no angular offset, for example tilt, pitch, roll, or yaw, relative to a gravitational force vector. The surgical orientation device 172 can store gravitational zero for calculations related to the table plane. In some methods, gravitational zero is registered only once and utilized throughout the procedure. The table registration can include recording a measurement of gravitational zero.

The table reference frame can include two perpendicular planes. The two perpendicular planes can include a vertical plane and a horizontal plane. The vertical plane can approximate the Anterior Pelvic Plane. The vertical plane may be recorded and stored by the surgical orientation device 172 and/or the orientation sensing device 204. The horizontal plane can approximate the plane of the table. The horizontal plane may be recorded and stored by the surgical orientation device 172 and/or the orientation sensing device 204.

The orientation of the vertical plane can be a baseline for placement of the cup portion of a hip prosthesis. For instance, abduction and anteversion angles can be calculated relative to the vertical plane. The system 600 can calculate cup angles relative to the vertical plane based on the assumption that the pelvis of the patient is correctly positioned such that the Anterior Pelvic Plane is vertical. In some embodiments, the vertical plane is determined by the surgical orientation device 172 and/or the orientation sensing device 204. In some methods of use, the abduction and anteversion angles in cup placement in total hip arthroplasty can be with respect to the vertical plane determined by table registration.

The surgical orientation device 172 can display information with respect to the calculated vertical plane. In some methods of use, the abduction and anteversion angles navigated during cup placement can be with respect to the vertical plane. In some embodiments, the surgical orientation device 172 and/or the orientation sensing device 204 can provide orientation and/or position data related to vertical plane.

In some embodiments, the vertical plane is a proxy for the Anterior Pelvic Plane for the posterior approach. The vertical plane can be determined completely independently of any anatomical landmarks. The vertical plane provides a reference plane that is unaffected by pelvic tilt. The vertical plane provides a reference plane that is unaffected by errors in landmark registration due to soft tissue.

As discussed herein, the method of use can include using the probe 678 and the sensor 204 to estimate a horizontal plane of the surgical table upon which the patient is resting. In some methods of use, the horizontal or table plane is calculated based on aligning the probe 678. As discussed herein, the method of use can include using the probe 678 and the sensor 204 to estimate a vertical plane utilizing a measurement of gravity. The system 600 can establish a reference frame for guiding the placement of the cup without registering landmarks. In some methods of use, the vertical plane is calculated based on a measurement of gravity. In some methods of use, target cup angles are relative to the vertical plane determined by gravity. In some methods of use, navigated cup angles are relative to the vertical plane which approximates the Anterior Pelvic Plane.

The pre-operative and/or post-operative images, such as x-rays, are captured within a reference plane similar to the Anterior Pelvic Plane. The table registration may provide a reference plane that approximates the reference plane of imaging techniques. The table registration may provide a reference plane that approximates the Anterior Pelvic Plane.

The system 600 can measure leg length and/or joint offset within the established reference frame. The system 600 can also compare the leg length between the two legs of the patient. The measurements of leg length and/or joint offset can be measured before and after cup placement. The measurements can be pre-operative and post-operative. In some methods of use, the surgeon can register a point. In some methods of use, the surgeon registers a single point on the femur. In some methods of use, the surgeon registers a single anatomical landmark during the procedure. The probe 678 can be coupled to the second assembly 606. The orientation sensing device 204 can be coupled to the probe 678 and/or the dock 662. The probe 678 can be immobilized to register the point. Once the tip of the distal end 680 is in contact with the desired point, the system 600 processes data from and stores the orientation of one or more sensor(s) in the orientation sensing device 204.

In some techniques, the table registration is completed prior to dislocating the hip. In some techniques, the point is registered prior to dislocating the hip. In some techniques, the incidence of light 180 is marked prior to dislocating the hip. Once registration is complete, the surgeon can proceed to dislocate the hip, resect the femoral head if applicable, and prepare the acetabulum as per the implant manufacturer's technique. The torque applied during dislocation can move pelvis away from the initial alignment.

In some methods of use, the surgical orientation device 172 can provide options to the user regarding how to proceed. In some methods of use, the surgeon can set target anteversion and/or abduction cup angles by inputting the target angles into the surgical orientation device 172. In some methods of use, the surgeon can set target anteversion and/or abduction cup angles by transferring the target anteversion and/or abduction cup angles from the external output device 130. In some methods of use, the surgeon can set target anteversion and/or abduction cup angles by transferring the target anteversion and/or abduction cup angles based on data collected by the module 100. In some methods of use, the surgeon can set target anteversion and/or abduction cup angles by adjusting population average angles based on patient specific factors. The range of motion detected by the module 100 can suggest increase or decrease of the patient specific anteversion angle from the population average anteversion angles. In some methods of use, the surgical orientation device 172 can store patient specific target cup angles determined in part by the module 100.

In some methods of use, the surgical orientation device 172 can provide options related to leg length and joint offset. In some methods of use, the surgical orientation device 172 can provide options related to leg length two the two legs of the patient. The measurements of leg length and/or joint offset can be recorded by the surgical orientation device 172 before and after cup placement. In some methods of use, the surgical orientation device 172 can measure leg length from a pre-operative state. In some methods of use, the surgical orientation device 172 can measure joint offset from a pre-operative state. In some methods of use, the surgical orientation device 172 can measure changes in the registered point.

The surgeon can remove the orientation sensing device 204 from the second assembly 606. The orientation sensing device 204 and the surgical orientation device 172 can be used to guide placement of the cup in the prescribed orientation. The surgeon can couple the orientation sensing device 204 to an impactor 300A. In some embodiments, the orientation sensing device 204 can be releasably coupled to a universal impactor adapter. The universal impactor adapter allows the orientation sensing device 204 to couple to any impactor.

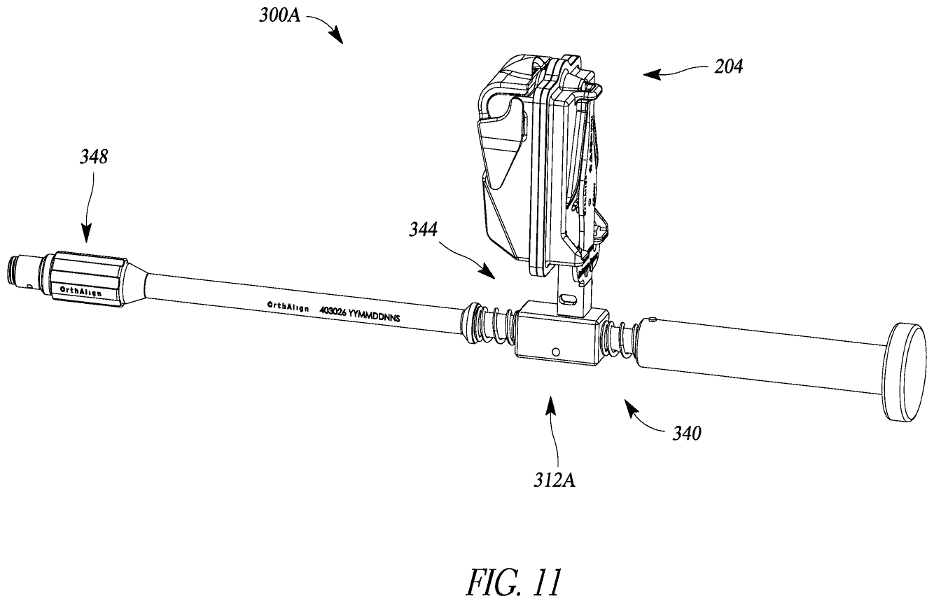

In some methods of use, the orientation sensing device 204 can be coupled to the impactor 300A as shown in FIG. 11. The orientation sensing device 204 can determine cup angles relative to a reference plane as the impactor 300A is moved. The impactor 300A can include a shell 312A. The movement of the shell 312A is cushioned by a plurality of spring members 340, 344 which are configured to absorb at least some of the shock of the impact on the impactor 300A. The shell 312A can include a coupler that permits the orientation sensing device 204 to couple to the shell 312A. The acetabular cup can be threaded onto a tip component 348. The user can select the appropriate cup adapter for the desired impactor. The orientation sensing device 204 can be coupled to the impactor 300A in order to navigate cup angles, as described herein.

The acetabular cup can be inserted into the acetabulum and positioned at the desired angle. As described herein, the surgeon can store patient specific target angles with the surgical orientation device 172. The surgical orientation device 172 can guide the surgeon in placing the cup relative to the patient specific target angles. As described herein, the target abduction and anteversion angles can be cup angles determined by pre-operative planning based on data from the module 100. The target abduction and anteversion angles from the pre-operative plan can be an input into the system 600.

In some methods of use, once the orientation sensing device 204 is attached to impactor 300A or any other impactor with the universal impactor adapter, the surgical orientation device 172 can display radiographic inclination and anteversion angles of impactor 300A. In some methods of use, the surgical orientation device 172 can display radiographic inclination and anteversion angles of impactor 300A relative to frontal pelvic plane. In some methods of use, the surgical orientation device 172 can display radiographic inclination and anteversion angles of impactor 300A relative to the vertical plane. In some methods of use, the surgical orientation device 172 can display radiographic inclination and anteversion angles of impactor 300A relative to a plane that approximates the Anterior Pelvic Plane. The angle of the impactor 300A can be calculated in real-time.

The surgical orientation device 172 can graphically display when the orientation sensing device 204 is navigated to the patient specific target abduction and anteversion angles. The surgical orientation device 172 can include indicia such as a target or bullseye, wherein the patient specific abduction and anteversion angles is located at the center of the target or bullseye. The surgical orientation device 172 can include indicia such as a dot or cross-hair to indicate movement of the impactor 300A relative to the target or bullseye. Aligning the indicia in the center of the target or bullseye can indicate that the impactor 300A is aligned with the patient specific target cup angles. In some methods of use, the surgeon aligns cross-hairs in a center of a bull's eye. In some methods of use, the surgeon aligns a bubble level. In some methods of use, the surgeon aligns two indicia. In some methods of use, the surgeon substantially aligns a moving indicia with a fixed indicia. In some methods of use, the surgeon moves an indicia on the graphical user interface of the surgical orientation device 172 by moving the impactor 300A. In some methods of use, the surgeon moves an indicia on the graphical user interface of the surgical orientation device 172 by moving the orientation sensing device 204. Aligning a visual indicator displayed on the surgical orientation device 172 can guide the user to position the impactor 300A at the desired patient specific target cup angles. The indicia can move in real-time.

The surgical orientation device 172 can graphically display the patient specific abduction and anteversion angles. As described herein, the patient specific abduction and anteversion angles can be an input for the system 600, determined in part by motion detected by one or more modules 100. In some methods of use, the indicia align when the impactor 300A is positioned at the patient specific target abduction angle. In some methods of use, the surgical orientation device 172 provides a graphical display to assist the surgeon in aligning the cup with the patient specific target angles. The surgical orientation device 172 can be coupled to the first assembly 606 during cup placement. The surgical orientation device 172 can be within the surgical field during cup placement. The surgical orientation device 172 can be coupled to the pelvis during cup placement.

In some methods of use, the surgical orientation device 172 displays the patient specific target angles determined by the pre-operative plan. The patient specific target abduction and anteversion angles can be displayed statically. In some methods of use, the surgical orientation device 172 displays a recommendation to the surgeon based on the pre-operative plan. The recommendation can include increasing the target anteversion angle from the population average. The recommendation can include decreasing the target anteversion angle from the population average. The recommendation can include using the population average as the patient specific target anteversion angle. As described herein, angles displayed can be calculated according to their radiographic definitions.

The surgical orientation device 172 can provide cup angles relative to any reference plane, including those described herein. The surgical orientation device 172 can provide cup angles relative to the vertical plane. The surgical orientation device 172 can provide cup angles relative to a plane that approximates the Anterior Pelvic Plane. The surgical orientation device 172 can provide cup angles relative to a plane acquired during table registration. The surgical orientation device 172 can provide cup angles relative to a plane that includes a vector for gravity.

In some embodiments, the surgeon can check cup angle after impacting as cup angle may change during impaction. In some embodiments, the cup angle is displayed up to the beginning of impaction, typically after the first mallet strike. In some methods of use, the surgeon will typically strike the impactor several times during cup placement. In some methods of use, these strikes may change the orientation of the cup. In some embodiments, the surgical orientation device 172 displays only the cup angle from prior to impaction. In some methods of use, the surgeon will repeat the calibration by coupling the orientation sensing device 204 to the system 600 such that the orientation sensing device 204 is in a fixed orientation relative to the surgical orientation device 172. Then the surgeon will move the orientation sensing device 204 back to the impactor 300A to check the cup angle. The system 600 can display the current cup angle after impaction. In some embodiments, the surgeon can visually confirm cup angles before proceeding.

After positioning the cup, the user can attach the second assembly 606 to the first assembly 604. In some methods of use, the user can measure for length and/or joint offset. In some methods of use, the user can measure both legs of the patient to determine leg length. In some embodiments, the surgeon can manually re-position the femur in the orientation prior to cup placement. In some methods of use, can register a point after cup placement. In some methods, measurements can be taken to compare the leg length before and after cup placement. In some methods, measurements can be taken to compare the joint offset before and after cup placement.

The method can include projecting light from the optical component 174 after cup placement. In some methods of use, the incidence of light 180 may not align with the marking after cup placement. In some methods of use, the femur has moved from the pre-operative orientation. In some methods, the surgeon positions the leg so that the optical component 174 projects a light onto the leg. In some embodiments, the surgeon can manually reposition the femur. The method can include repositioning the femur by aligning incidence of light with the prior marking or recording of the incidence of the light 180. In some embodiments, the surgeon can realign the femur such that the femur is in same positon before and after cup placement.

In some methods of use, the surgeon can register the point after repositioning the femur. The surgeon can position the distal end 680 of the probe 678 at the point. The orientation of the orientation sensing device 204 and the extension of the probe 678 can be input into the surgical orientation device 172. These data enable the surgical orientation device 172 to output amounts of change in leg length and leg offset. Once the tip of the distal end 680 is in contact with the desired point, the system 600 processes data from one or more sensor(s) in the orientation sensing device 204 to determine changes from the measurement taken before cup placement. The surgical orientation device 172 can display changes in leg length and/or leg offset.

### 3. Methods for Anterior Approach

FIG. 7 illustrate hip navigation system 600 which can be adapted to navigate a hip joint procedure from an anterior approach.

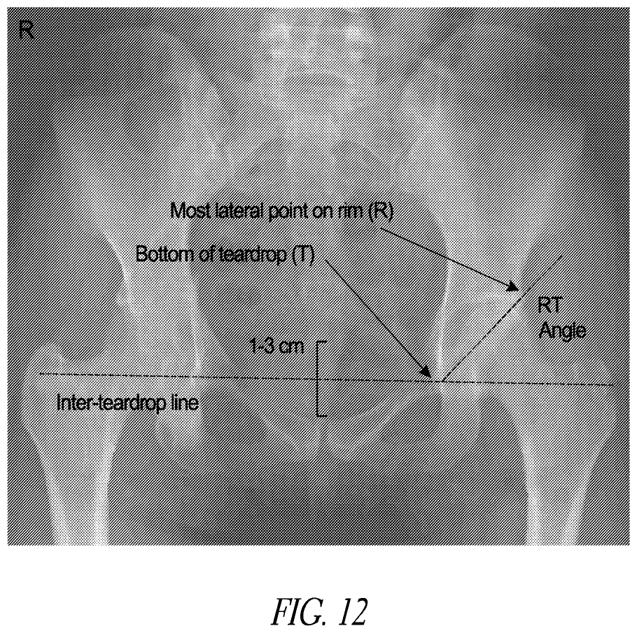

In some embodiments, the navigation system 600 is configured to locate a relevant anatomical feature to aid in proper placement of a prosthetic hip joint. In some methods, pre-operative imaging techniques are used. In some methods of use, the surgeon can use a standing or supine anteroposterior (AP) pelvic x-ray. FIG. 12 shows standing AP radiograph taken with patient standing with feet in neutral rotation and shoulder width apart in stance. The x-ray tube-to-film distance should be 120 cm, with the crosshairs centered on the midpoint between the superior border of the pubic symphysis and a line drawn connecting the anterior superior iliac spines (ASIS). The coccyx should be centered in line with the pubic symphysis, and the iliac wings, obturator foramina and radiographic teardrops should be symmetrical in appearance. For appropriate pelvic inclination or abduction, a 1-3 cm gap should be seen between the tip of the coccyx and the superior border of the pubic symphysis. This positioning can be important for measuring the patient specific Rim Teardrop (RT) angle.

To obtain the patient specific Rim Teardrop (RT) angle from the AP pelvic x-ray the surgeon can complete one or more of the following steps. The surgeon can draw a line on the x-ray connecting the bottom of the teardrops. The surgeon can draw a line from the most lateral point on the rim of the acetabulum (R) on the operative side through the bottom of the teardrop (T) to the horizontal inter-teardrop line. If osteophytes are present on the rim (R), the surgeon can draw a line through the most lateral osteophyte. The surgeon can measure the angle between the inter-teardrop line and the RT line just drawn. This patient specific RT abduction angle can be an input for the system 600.

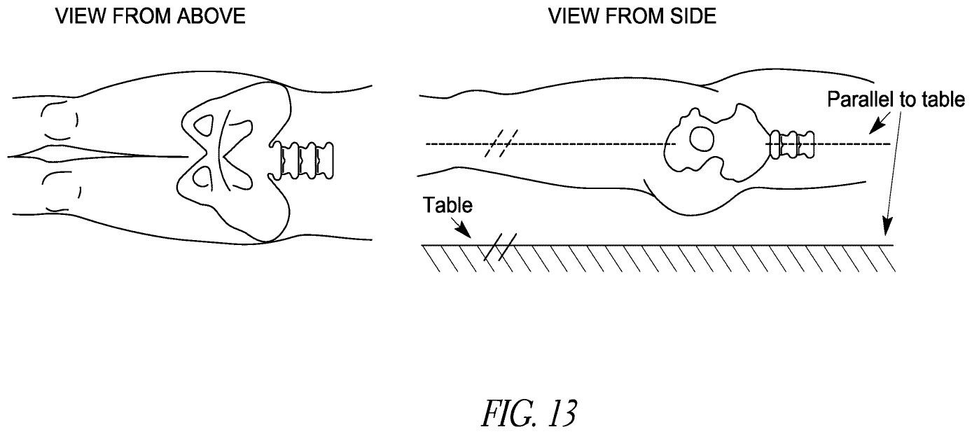

FIG. 13 shows the patient positioning for the anterior hip approach. In the anterior hip approach, the patient should be placed in the supine position. When positioning the patient prior to surgery, the surgeon should take care to align the spine and femur of a patient in a horizontal plane parallel to the long edge of the operating table. The surgeon can verify that patient is positioned in an appropriate position, e.g., in a supine position.

The system 600 can be partially assembled for calibration, as described herein. The orientation sensing device 204 can be fixed in position relative to the surgical orientation device 172. The surgical orientation device 172 and the orientation sensing device 204 can be calibrated.

The system 600 can be attached to the pelvis as shown in FIG. 7. In some methods, the fixation pins 610, 612 are placed in the ipsilateral iliac crest in parallel. The fixation pins 610, 612 are placed in a similar manner to the placement of pins for pelvic external fixation. The fixation pins 610, 612 enter the iliac wing at its most superior surface and travel between the tables of the inner and outer bone of the iliac wing. In order to prevent obstruction of subsequent femoral exposure and broaching with the anterior approach, in some methods of use, the most anterior fixation pin should be placed 2-4 cm posterior to the ASIS. In order to prevent obstruction of subsequent femoral exposure and broaching with the anterior approach, in some methods of use, the most anterior fixation pin should be placed 2-4 cm posterior to the ASIS. In some techniques, one the fixation pins 610, 612 is positioned on the iliac crest 2-4 cm posterior to ASIS.

The surgeon can register a parked configuration or home position. In some techniques, the distal end 680 of the probe 678 can be engaged with a point on the fixation base 602. The probe 678 can be vertical in the home position. The orientation sensing device 204 can be vertical in the home position.

The surgeon can position the distal end 680 of the probe 678 at various anatomical landmarks or points. The landmarks include the two anterior superior iliac spines (ASIS) which are bony projections of the iliac bone. The anterior superior iliac spines can be visualized and/or palpitated by the surgeon during surgery. The anterior superior iliac spine is the anterior extremity of the iliac crest of the pelvis. The inter-ASIS line extends between these landmarks. The inter-ASIS line extends between the ipsilateral ASIS and the contralateral ASIS. In other methods of use, other landmarks are used. Other landmarks that could be used include locations on the ilium, ischium, pubis, anterior insertion point of trans-acetabular ligament to the ischium, mid-point of the inferior aspect of the acetabular notch, the anterior superior iliac spine, anterior inferior iliac spine, convergence of the acetabulum and anterior inferior iliac spine, as well as the other landmarks known in the art.

The system 600 has one or more processors that receive(s) data and determines the relative position and/or orientation of these anatomical landmarks when the probe 678 contacts the anatomical landmark. The data can be generated by inertial sensors, as discussed elsewhere herein, or other types of sensors of the system 600. Preferably the sensors are small enough to be mounted on or in handheld housings or embedded in the instruments, such as the surgical orientation device 172 and the orientation sensing device 204. The system 600 preferably also has a memory device to at least temporarily store the position of these points. The system 600 preferably also has the ability to at least temporarily store the relevant position and/or orientation data when the probe 678 contacts the anatomic landmarks. In some methods of use, the system 600 records the position and/or orientation of the probe 678 when the probe 678 contacts each anatomic landmark or point.

In some methods of use, the user can measure for leg length and joint offset. At the surgeon's discretion, the system 600 can be used to navigate a condition, location and/or orientation of the femur prior to hip replacement. In some embodiments, a mark Fm may be made on the proximal femur. In some embodiments, a structure is attached to the femur, such as a pin. In some embodiments, a burr or detent is made in the femur. The distal end 680 of the probe 678 can be brought into contact with the femur mark Fm. The surgical orientation device 172 can be signaled to record the orientation of the orientation sensing device 204. In some methods of use, the system 600 includes an optical component 174. The laser light can be used to project a point, a plane, and or a cross-hair onto a target or targets, including but not limited to an anatomical feature or landmark. The surgeon can mark one or more points along the line of the incidence of light 180 of the optical component 174.

a. Anterior Pelvic Plane

The system can calculate the Anterior Pelvic Plane. The Anterior Pelvic Plane can be defined as a plane created by the two anterior superior iliac spines (ASIS) and the anterior surface of the pubic symphysis. Three points provide adequate information to calculate a plane. These anatomical features are visible and/or palpable while the patient is in a supine position. In some methods, the probe 678 registers the Anterior Pelvic Plane with direct contact of anatomical landmarks when the patient is in the supine position. The system 600 is then able to provide navigation data of the orientation of a hip instrument (e.g., the impactor 300A or any impactor with universal impactor adapter) with respect to the Anterior Pelvic Plane. In some embodiments, the system 600 is able to provide the user navigation data in real-time. In the anterior approach, the patient is positioned on his/her back and the Anterior Pelvic Plane is oriented substantially horizontally, e.g., substantially parallel to the plane of the table on which the patient is positioned.

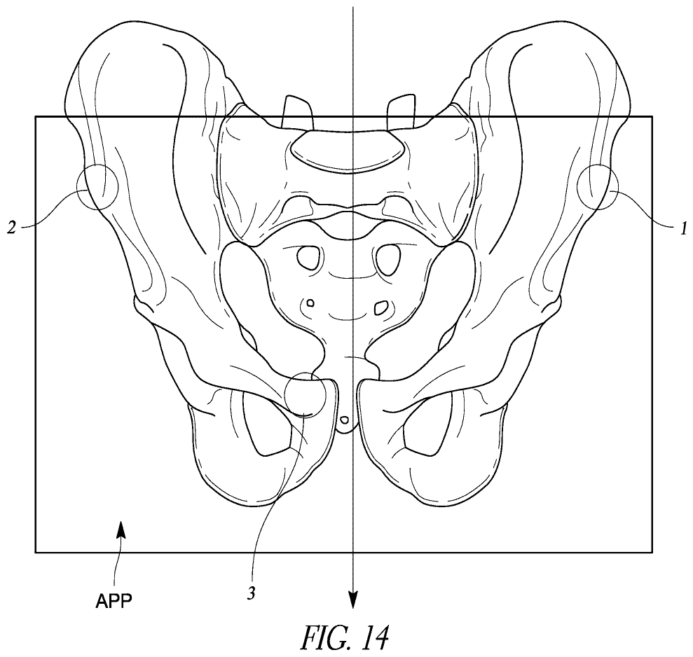

The system 600 can determine the Anterior Pelvic Plane by registering Point 1, Point 2, and Point 3 as shown in FIG. 14. The surgeon can register Point 1, Point 2, and Point 3. The illustrated embodiment shows Point 1 on the left hip, Point 2 on the right hip, and Point 3 on the right hip of the patient. In some methods, Point 1 is the ipsilateral ASIS. In some methods, the distal end 680 of the probe 678 is placed at the ipsilateral ASIS landmark. The probe 678 can be immobilized and the position and/or orientation of the orientation sensing device 204 can be recorded by the surgical orientation device 172. The surgeon can enter an input to register Point 1 (e.g., depress a button on the surgical orientation device 172). The surgical orientation device 172 can indicate that Point 1 was recorded. Additionally, the distance that the probe 678 is extended to contact the ipsilateral ASIS, as captured by the camera of the orientation sensing device 204, can be recorded by the orientation device 172.

In some methods, Point 2 is the contralateral ASIS. In some methods, the distal end 680 of the probe 678 is placed at the contralateral ASIS landmark. The probe 678 can be immobilized and the position and/or orientation of the orientation sensing device 204 can be recorded by the surgical orientation device 172. The surgeon can enter an input to register Point 2 (e.g., depress a button on the surgical orientation device 172). The surgical orientation device 172 can indicate that Point 2 was recorded. Additionally, the distance that the probe 678 is extended to contact the contralateral ASIS, as captured by the camera of the orientation sensing device 204, can be recorded by the orientation device 172.

In some methods, Point 3 is the anterior surface of the pubic symphysis. In some methods, the distal end 680 of the probe 678 is placed at the pubis landmark. In some methods, either pubic tubercle may be used as Point 3. In some embodiments, the contralateral pubic tubercle is used as Point 3. The probe 678 can be immobilized and the position and/or orientation of the orientation sensing device 204 can be recorded by the surgical orientation device 172. The surgeon can enter an input to register Point 3 (e.g., depress a button on the surgical orientation device 172). The surgical orientation device 172 can indicate that Point 3 was recorded. Additionally, the distance that the probe 678 is extended to contact the pubic symphysis, as captured by the camera of the orientation sensing device 204, can be recorded by the orientation device 172. The process to record points can be repeated for one or more additional points.

The distance related to the extension of the probe 678 can be used in conjunction with the positional and/or orientation data from the orientation sensing device 204. In some methods, the surgical orientation device 172 and/or the orientation sensing device 204 converts the image of the camera 184 into an extension measurement of the probe 678. When registering the anatomical points, the camera can capture an image of the markings of the probe 678. The system 600 can provide an accurate determination of the translational position of the probe 678. The system 600 use the length measurement and the data from the orientation sensing device 204 to determine the location of the distal end 680 of the probe 678 at point 1, 2, and 3.

Once the foregoing points of the pelvis have been navigated and the data recorded into the surgical orientation device 172, the Anterior Pelvic Plane can be calculated from data of the navigated points. The system 600 can calculate the Anterior Pelvic Plane from the three points recorded by the system 600. The three points are shown in FIG. 14. The inter-ASIS line connects Point 1 and Point 2. The inter-ASIS line provides a straight line between the ipsilateral ASIS and the contralateral ASIS. The Anterior Pelvic Plane can be considered a horizontal plane that pivots about the inter-ASIS axis depending on the location of Point 3. FIG. 14 illustrates the Anterior Pelvic Plane (APP) which is determined by Point 3. The Anterior Pelvic Plane includes the ipsilateral ASIS, the contralateral ASIS, and the anterior surface of the pubic symphysis.

In some methods of use, the orientation of the Anterior Pelvic Plane can be a baseline for placement of the cup portion of a hip prosthesis. The surgical orientation device 172 can display information with respect to the Anterior Pelvic Plane. In some methods of use, the abduction and anteversion angles in cup placement in total hip arthroplasty can be with respect to the Anterior Pelvic Plane. In some embodiments, the Anterior Pelvic Plane is determined by the surgical orientation device 172 and/or the orientation sensing device 204.

b. Adjusted Pelvic Plane

In some methods of use, the reference plane is adjusted. In some methods of use, the table plane is estimated to adjust the reference plane. Any method or method step to establish the table plane described herein can be utilized. The table plane can be a horizontal or generally horizontal plane. The patient can be positioned so that the coronal plane of the pelvis is level, e.g., parallel to the table and/or also horizontal. The user can visually verify the coronal plane is level or can use devices to position the patient's body to align the coronal plane with the plane of the top surface of the table. In some embodiment, a reference plane based on the plane of the top surface of the table can be input into the system 600. The table registration provides clinical value. In some techniques, the table plane can be an input for a secondary reference plane to the Anterior Pelvic Plane.

During table registration, the probe 678 is coupled with the orientation sensing device 204. In some methods of use, the probe 678 and the orientation sensing device 204 are constrained together in movement during table registration. In some embodiments, the probe 678 can be uncoupled with other components of the first assembly 604 and/or the second assembly 606. In some methods of use, the system 600 is assembled, such that the first assembly 604 and the second assembly 606 are coupled to the patient via the fixation pins 610, 612. In some embodiments, the probe 678 can be coupled the first assembly 604, the second assembly 606, and/or the pelvis during table registration.

The probe 678 can be aligned with the long axis of the body. The probe 678 can be held substantially parallel to the plane of the table. The surgeon can visually inspect the probe 678 from one or more locations. For instance, the user can inspect the probe 678 from a top view and a side view.

The probe 678 can be held steady and the position and/or orientation of the orientation sensing device 204 can be recorded by the surgical orientation device 172. The user can enter an input to register the table (e.g., depress a button on the surgical orientation device 172). The user can interact with a user interface on the surgical orientation device 172 to signal to the surgical orientation device 172 to capture the position and/or orientation of the orientation sensing device 204. The surgical orientation device 172 can indicate that the table plane was recorded.

As described herein, the orientation sensing device 204 and the surgical orientation device 172 can include one or more inertial sensors. The one or more inertial sensors can detect gravity and provide a vector for the down direction. The table plane provides an estimation of the orientation of the coronal plane.

In some embodiments, the method can include calculating an Adjusted Plane (ADJUSTED). The inter-ASIS line connects Point 1 and Point 2, as discussed herein. The inter-ASIS line provides a straight line between the ipsilateral ASIS and the contralateral ASIS. As described herein, Point 1 and Point 2 can be recorded by the system 600. Point 1 and Point 2 can be recorded as part of a method to calculate the Anterior Pelvic Plane. Point 1 and Point 2 can be recorded independently from a method to calculate the Anterior Pelvic Plane. In some methods, Point 1 is the ipsilateral ASIS. In some methods, Point 2 is the contralateral ASIS. These anatomical features are visible and/or palpable while the patient is in a supine position. The Adjusted Plane can be calculated in addition, or as an alternative, to the Anterior Pelvic Plane. The system 600 is able to provide the user navigation data of the orientation of a hip instrument (e.g., the impactor 300A or any impactor with the universal impactor adapter) with respect to the Adjusted Plane.

The Adjusted Plane is determined by a rotation of the Anterior Pelvic Plane about the inter-ASIS line to be perpendicular to the force of gravity. The Adjusted Plane utilizes a measurement of gravity. As described herein, the surgical orientation device 172 comprises one or more inertial sensors. As described herein, the orientation sensing device 204 comprises one or more inertial sensors. In some embodiments, inertial data from one or more inertial sensors is used to calculate the Adjusted Plane. The surgical orientation device 172 and/or the orientation sensing device 204 can comprise an accelerometer, which can provide a measurement of the direction of gravity. The one or more inertial sensors can provide a vector aligned with vertical, e.g., for the down direction. The accelerometer can produce a horizontal vector by transforming the vertical vector (e.g., by rotation of 90 degrees). The accelerometer can provide orientation and/or position data such that the Adjusted Plane is perpendicular to the force of gravity.

The surgical orientation device 172 and/or the orientation sensing device 204 can provide a reference to gravitational zero. Gravitational zero, as referred to herein, refers generally to an orientation in which an axis of a sensor is perpendicular to the force of gravity, and thereby experiences no angular offset, for example tilt, pitch, roll, or yaw, relative to a gravitational force vector.

In some methods of use, the orientation sensing device 204 can be positioned in other ways than horizontally to measure the direction of gravity. In some methods of use, the orientation sensing device 204 can measure gravity when in the home position. In some methods of use, the orientation sensing device 204 can be positioned vertically or substantially vertically to measure gravity. In some methods of use, the orientation sensing device 204 can measure gravity when contacting a point or anatomical landmark. In some methods of use, the orientation sensing device 204 can measure gravity when contacting Point 1. In some methods of use, the orientation sensing device 204 can measure gravity when contacting Point 2.

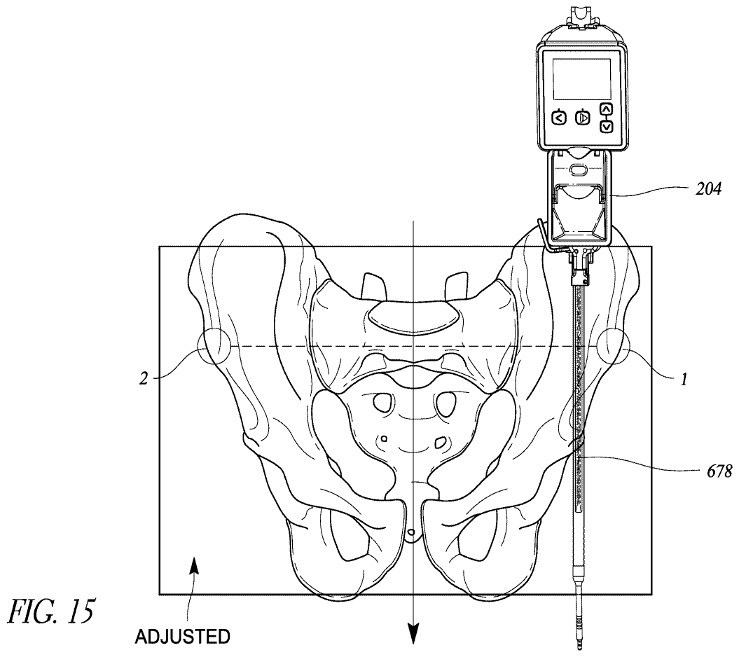

FIG. 15 illustrates the Adjusted Plane. The Adjusted Plane can be considered a horizontal plane that pivots about the inter-ASIS line depending on the measurement of gravity. Once the foregoing points of the pelvis have been navigated and the data recorded into the surgical orientation device 172, the Adjusted Plane can be calculated from data for the navigated points and the gravity measurement. The Adjusted Plane includes the ipsilateral ASIS and the contralateral ASIS. The Adjusted Plane includes the inter-ASIS line. The system 600 can combine the horizontal vector with Point 1 and Point 2 to calculate the Adjusted Plane. In some embodiments, the Adjusted Plane can be considered a horizontal plane determined by the direction of gravity. In some embodiments, the Adjusted Plane can be considered a horizontal plane containing the inter-ASIS line.

As described herein, three anatomical landmarks are registered for the Anterior Pelvic Plane. The ipsilateral ASIS, the contralateral ASIS, and the pubic symphysis, as registered by the probe 678, define the Anterior Pelvic Plane. As described herein, two anatomical landmarks are registered for the Adjusted Plane. The two landmarks define the inter-ASIS line.

In some embodiments, the surgical orientation device 172 can provide a corrected reference frame by transforming, e.g., by rotating the Anterior Pelvic Plane about the inter-ASIS line based on the direction of gravity. In some embodiments, the surgical orientation device 172 can rotate the Anterior Pelvic Plane about the inter-ASIS line to provide a corrected reference frame that is aligned with horizontal. The measurement of gravity enables the surgical orientation device 172 to rotate the Anterior Pelvic Plane. The one or more inertial sensors of the orientation sensing device 204 and/or the surgical orientation device 172 can provide data to determine how to transform, e.g., to rotate, a plane containing the inter-ASIS axis to be perpendicular to the force of gravity.

The Adjusted Plane provides a check against gross errors from the anatomical registrations. The Adjusted Plane provides a reference plane that is unaffected by pelvic tilt. The Adjusted Plane provides a reference plane that is largely unaffected by errors in registration due to soft tissue. The Adjusted Plane is calculated, in part, on properly aligning the probe 678 with the table or other horizontal plane. The Adjusted Plane provides a horizontal plane which may be useful for comparing the navigated cup angles to pre-operative and/or post-operative images. The pre-operative and/or post-operative images, such as x-rays, are captured within a horizontal reference plane. The Adjusted Plane may provide a horizontal reference plane that approximates the horizontal reference plane of imaging techniques.

The orientation of the Adjusted Plane can be a baseline for placement of the cup portion of a hip prosthesis. The surgical orientation device 172 can display information with respect to the Adjusted Plane. In some methods of use, the patient specific abduction and anteversion angles in cup placement in total hip arthroplasty can be with respect to the Adjusted Plane.

c. Cup Placement

Once registration is complete, the user can proceed to proceed to dislocate the hip, resect the femoral head, and prepare the acetabulum. The surgeon can prepare the impactor 300A and acetabular cup. The surgeon can couple the orientation sensing device 204 to the impactor 300A.

The surgeon can select a patient specific target cup angle based on the preoperative plan. The acetabular cup can be inserted into the acetabulum and positioned at the desired angle. The surgical orientation device 172 can guide the surgeon in navigation to the patient specific target cup angles. The surgical orientation device 172 can graphically display when the orientation sensing device 204 is navigated to the patient specific abduction and anteversion angles. The patient specific abduction and anteversion angles can be pre-operative cup angles determined by the module 100. The surgical orientation device 172 can graphically display the abduction and anteversion angles as the orientation sensing device 204 and the impactor 300A are moved.

The surgical orientation device can provide cup angles relative to any reference plane, including those described herein including the Anterior Pelvic Plane and the Adjusted Plane. The acetabular shell can be inserted into the acetabulum and positioned at the patient specific target angle. The surgical orientation device 172 can guide the surgeon in navigating to the appropriate cup angle. The surgical orientation device 172 can graphically display when the orientation sensing device 204 is navigated to the patient specific target abduction and anteversion angles. The desired abduction and anteversion angles can be pre-operative cup angles based in part on the module 100. The surgical orientation device 172 can graphically display the patient specific target abduction and anteversion angles as the orientation sensing device 204 is moved. The surgeon can align impactor 300A at desired cup angle. Aligning a visual indicator of the surgical orientation device 172 can guide the user to position the impactor 300A at the desired cup angles. The patient specific abduction and anteversion angles can be displaced statically or dynamically. The cup angles can be checked after impacting by repeating one or more method steps described herein.

The surgical orientation device 172 can include indicia such as a target or bullseye to indicate the patient specific target abduction and anteversion angles. The surgical orientation device 172 can include indicia such as a dot or cross-hair to indicate movement of the impactor 300A. Aligning the indicia in the center of the target or bullseye can indicate that the impactor 300A is aligned with the patient specific target cup angles. The indicia can move in real-time. The method for cup placement and verification can include any steps described herein.

If femoral landmark Fm is acquired in the procedure prior to separating the natural joint, the same landmark can be acquired after the cup is placed to confirm that the cup placement has not changed either the length of the leg, the off-set of the leg from the trunk of the patient or both. The distal end 680 of the probe 678 can be brought into contact with the same landmark (e.g., Fm) acquired early in the procedure. The orientation of the orientation sensing device 204 and the extension of the probe 678 can be input into the surgical orientation device 172. This data can enable the surgical orientation device 172 to output amounts of change in leg length and/or leg offset.

The optical component 174 can provide a visual guide to replicate the original position of the femur relative to the pelvis after cup placement. When measuring changes in leg length and lateral joint offset, the apparent changes are sensitive to changes in the orientation of the femur relative to the pelvis. The user can reposition the femur prior to measuring the change in leg length and joint offset such that the orientation of the femur relative to the pelvis is the same as that when the preoperative baseline measurement was made.

### 4. Adjusted Plane and Anterior Pelvic Plane Comparison

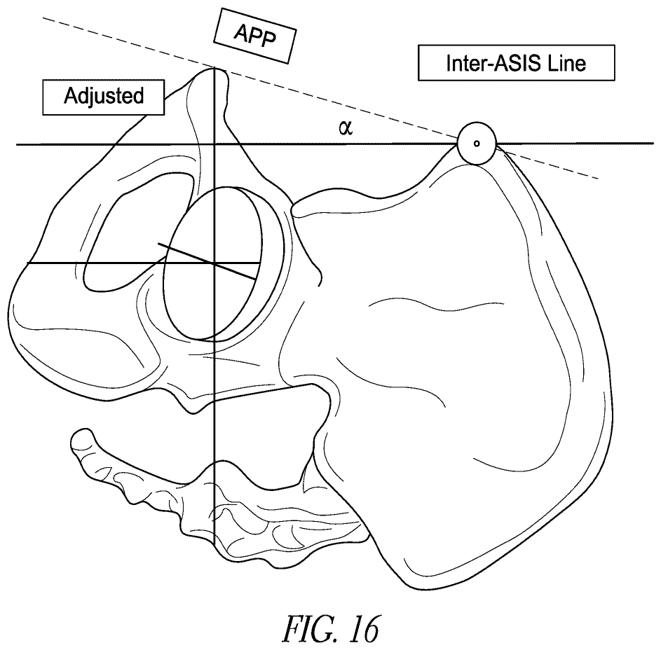

FIG. 16 illustrates a comparison between the Anterior Pelvic Plane and the Adjusted Plane. As described herein, the Anterior Pelvic Plane and the Adjusted Plane both contain the inter-ASIS line. The Anterior Pelvic Plane also contains Point 3 to define the plane. The Adjusted Plane utilizes the direction of gravity to define the plane. In some embodiments, the Anterior Pelvic Plane and the Adjusted Plane are coaxial about the inter-ASIS line. The Adjusted Plane is a rotation of the Anterior Pelvic Plane about the inter-ASIS line. The Adjusted Plane and the Anterior Pelvic Plane can form an angle alpha. The angle alpha can be a measurement of pelvic tilt. The angle alpha can measure the difference to horizontal from the Anterior Pelvic Plane. The angle alpha can adjust the Anterior Pelvic Plane such that the cup angles more closely match those shown in post-operative x-rays. In some embodiments, the Adjusted Plane adjusts for tilt. In some embodiments, the Adjusted Plane adjusts for tilt of the pubis relative to the inter-ASIS line.

The Adjusted Plane has clinical value. The pre-operative and post-operative x-rays produce two-dimensional images on a horizontal plane. The image receptor is positioned horizontally relative to the patient in the supine position. The image receptor captures the remnant beam as the beam exits the body of the patient. The anatomy of the patient is projected onto the horizontal plane of the image receptor. For pelvis x-rays, the routine projection is an anterior-posterior projection. The navigate angles can be correlated to the view of the post-operative x-ray via the Adjusted Plane. The Adjusted Plane may provide a horizontal reference plane that approximates the horizontal reference plane of imaging techniques. There is a clinical benefit in providing users with navigated cup angles during a procedure that will reflect those measured on post-operative x-rays. The navigated cup angles based on the Adjusted Plane can be provided in addition, or as a substitute to those based on the Anterior Pelvic Plane. The navigated cup angles based on the Adjusted Plane can be provided in addition, or as a substitute to those based on the table plane.

The adjustment from the Anterior Pelvic Plane to the Adjusted Plane can provide the user with cup angles that correlate to clinically expected cup angles. The adjustment from the Anterior Pelvic Plane to the Adjusted Plane can provide the user with cup angles that correlate to what they expect to see during surgery (e.g., a comparison between the image of the cup and the output of the system 600). The adjustment from the Anterior Pelvic Plane to the Adjusted Plane can provide the user with cup angles that correlate to what they expect to see on post-operative images (e.g., a comparison between the output of the system 600 and post-operative images). The user, such as a surgeon, correlates the output of the system 600 with numbers that correlate to clinical experience, e.g., calibrated to what they expect to see in post-operative images. The post-operative images can be 6 week post-operative supine films. The post-operative images are taken from a horizontal cross-section of the patient's body. The Adjusted Plane may be more similar to the plane of the post-operative images.

For the system 600, the surgeon is provided with an output that assists a surgeon to navigate to proper cup placement. The output can be abduction and anteversion angles displayed on the surgical orientation device 172. The user can navigate to the desired abduction and anteversion angles by moving components of the system 600, such as the impactor 300A. When the surgeon places the cup at the desired abduction and anteversion angles, the user wants to see the same or similar angle on post-operative images. The user's confidence in the navigation of the system 600 increases if the abduction and anteversion angles produced by the system 600 match the post-operative images.

Systems and methods described herein can improve prosthetic hip joint placement using navigation in combination of pre-operative imaging and landmark referencing. These hip procedures generally guide a prosthetic hip to an orientation within the acetabulum that minimizes the chance of dislocation due to impingement of the femoral neck on the cup or on bones around the acetabulum or other reasons related to suboptimal orientation of the prosthetic. Various techniques leverage population averages of proper placement while others are amenable to patient specific refinements.

### C. Post-Operative Planning Systems for Anterior and Poster Approach

### 1. Post-Operative Planning System

A variety of pelvic mobility modules, systems, and methods for total hip arthroplasty (THA) are discussed below that can be used to improve outcomes for patients by increasing the likelihood of proper placement of a medical prosthesis. These systems and methods can be focused on understanding the patient's anatomy. These systems and methods can additionally or alternatively measure, quantify and/or track pelvic mobility post-operatively to track recovery or compliance.

The inertial measurements can be taken post-operatively for various reasons including to monitor recovery and improve compliance with physical therapy. To measure pelvic mobility, the user moves through a range of motion, wherein data is captured and processed. The patient can take home the module 100 after surgery. The module 100 can allow for post-operative functions. In some embodiments, the module 100 can track physical therapy. In some embodiments, the module 100 can track patient compliance. In some embodiments, the module 100 can be an informational or educational tool. Data from the module 100 can be transmitted to the external output device 130. Data from the module can be transmitted to the external memory 128. Data can be processed and analyzed, as described herein.

Although these inventions have been disclosed in the context of certain preferred embodiments and examples, it will be understood by those skilled in the art that this application extends beyond the specifically disclosed embodiments to other alternative embodiments and/or uses of the invention and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or sub-combinations of the specific features and aspects of the embodiments may be made and still fall within the scope of the application. For example, the application contemplates the connection hub alone or in combination with any of the other modules could comprise a separate aspect. Or, any one or a combination of the modules could be directly connected to an umbrella hub or overhead support to form another separate aspect. Accordingly, it should be understood that various features and aspects of the disclosed embodiments can be combined with or substituted for one another in order to form varying modes of the disclosed embodiments. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

1-112. (canceled)

113. A system comprising:
a module comprising one or more inertial sensors configured to be positioned relative to the anatomy of a patient, wherein the module is configured to determine one or more characteristics of the patient; and
an external output device.

114. The system of claim 113, wherein the one or more inertial sensors comprise an accelerometer.

115. The system of claim 113, wherein the one or more inertial sensors comprise a gyroscope.

116. The system of claim 113, wherein the module is configured to transmit data from the module to the external output device.

117. The system of claim 113, wherein the external output device comprises a tablet.

118. The system of claim 113, wherein the module is configured to measure a characteristic of movement.

119. The system of claim 113, wherein the module is configured to measure a characteristic of positioning of the patient.

120. The system of claim 113, wherein the module comprises a light.

121. The system of claim 113, wherein the module comprises a button.

122. A method of determining one or more characteristics of a patient comprising:
positioning a first module relative to a first portion of the anatomy of the patient, wherein the first module comprises one or more inertial sensors; and
positioning a second module relative to a second portion of the anatomy of the patient, wherein the second module comprises one or more inertial sensors.

123. The method of claim 122, wherein positioning the first module comprises positioning the first module relative to a pelvis of the patient.

124. The method of claim 122, wherein positioning the first module comprises positioning the first module relative to an ASIS point of the patient.

125. The method of claim 122, wherein positioning the first module comprises positioning the first module relative to a pubis of the patient.

126. The method of claim 122, wherein positioning the first module comprises positioning the first module relative to a femur of the patient.

127. The method of claim 122, wherein positioning the first module comprises positioning the first module relative to a tibia of the patient.

128. The method of claim 122, wherein positioning the first module comprises positioning the first module relative to a bony prominence identified by palpation of the skin of the patient.

129. The method of claim 122, wherein positioning the first module comprises positioning the first module relative to a palpable location of the patient.

130. The method of claim 122, wherein the one or more inertial sensors of the first module comprise an accelerometer.

131. The method of claim 122, wherein the one or more inertial sensors of the first module comprise a gyroscope.

132. The method of claim 122, further comprising transmitting data from the first module to an external output device.
